SEQUENCE LISTING (12 sequences)

SEQ ID NO: 1 - protein, 24 aa, Artificial Sequence
  Sequence derived from BRCA1
GAACGGGCTTGGAAGAAAATAATC

SEQ ID NO: 2 - protein, 19 aa, Artificial Sequence
  Sequence derived from BRCA1
TCAAGGGCAGAAGAGTCAC

SEQ ID NO: 3 - protein, 26 aa, Artificial Sequence
  Sequence derived from BRCA1
GCCTGTCGATTATACAGATGATGAAA

SEQ ID NO: 4 - protein, 21 aa, Artificial Sequence
  Sequence derived from BRCA1
CGCTGCCCAGTGTTCATTACT

SEQ ID NO: 5 - protein, 22 aa, Artificial Sequence
  Sequence derived from BRCA1
TTCTATCCTAATACCATCGACG

SEQ ID NO: 6 - protein, 22 aa, Artificial Sequence
  Sequence derived from BRCA1
TTTGAGTAGTCAAAGTCAGAGC

SEQ ID NO: 7 - protein, 20 aa, Artificial Sequence
  Sequence derived from BRCA1
CTCAACATCTCCCCCTTCTC

SEQ ID NO: 8 - protein, 20 aa, Artificial Sequence
  Sequence derived from BRCA1
CAAATCCCATATCCTCGTCC

SEQ ID NO: 9 - protein, 5 aa, Artificial Sequence
  Sequence derived from BRCA1
RXRXX

SEQ ID NO: 10 - protein, 14 aa, Artificial Sequence
  Sequence derived from BRCA1
TSKRHDSDTFPELK

SEQ ID NO: 11 - protein, 14 aa, Artificial Sequence
  Sequence derived from BRCA1
TSKRHDSDTFPELK

SEQ ID NO: 12 - protein, 1863 aa, Homo sapiens
  BRCA1
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

(19) United States
(12) Patent Application Publication
(10) Pub. No.: US 2012/0040896 A1
(43) Pub. Date: Feb. 16, 2012

(54) COMPOSITIONS, METHODS AND USES FOR MODULATION OF BRCA 1

(75) Inventors: Jeffrey Thomas Holt, Aurora, CO (US); Steve Anderson, Denver, CO (US); Andrew Cook Nelson, St. Paul, CO (US)

(73) Assignee: THE REGENTS OF THE UNIVERSITY OF COLORADO, Denver, CO (US)

(21) Appl. No.: 12/937,217

(22) PCT Filed: Apr. 13, 2009
(86) PCT No.: PCT/US09/40391

§ 371 Date: May 2, 2011

Related U.S. Application Data
(60) Provisional application No. 61/123,991, filed on Apr. 11, 2008.

(51) Int. Cl. A61K 38/17; A61K 38/30; A61P 35/00; A61K 31/404; A61K 31/663; C07K 16/18; A61K 38/28

(52) U.S. Cl. 514/5.9; 530/387.9; 514/8.6; 514/19.3; 514/414; 514/102; 514/19.4; 514/19.5

(57) ABSTRACT

Embodiments of the present invention generally relate to methods, compositions and uses for diagnosis and treatment of cancer. Certain embodiments report methods and compositions for diagnosing and/or treating a subject having a BRCA1-related cancer or sporadic cancer. Some embodiments disclose treatments that can include, but are not limited to, modulation of BRCA1. In some embodiments, methods for identifying a subject with unstable BRCA1 protein are reported.

### CROSS REFERENCE TO RELATED APPLICATIONS

This application claims the priority benefit of provisional U.S. patent application Ser. No. 61/123,991 filed on Apr. 11, 2008. The aforementioned application is incorporated by reference in its entirety for all purposes.

### FIELD

In some embodiments, methods, compositions and uses for diagnosis and treatment of cancer in a subject are reported. Certain embodiments disclose methods and compositions for diagnosing and/or treating a subject having a BRCA1-related cancer or sporadic cancer. Some embodiments reporting treatment can include, but are not limited to, modulation of BRCA1.

### BACKGROUND

Genetic analysis of familial breast and ovarian cancer indicate that BRCA1 is a tumor suppressor gene. Although mutations of BRCA1 are rare in sporadic cancers, some cases exhibit decreased BRCA1 mRNA expression suggesting that its loss may contribute to tumorigenesis in a proportion of non-hereditary cancers as well.

### SUMMARY

Embodiments of this application generally relate to methods, compositions and uses for modulations of BRCA1. In some embodiments, methods, compositions and uses for diagnosis and treatment of cancer in a subject are disclosed. Certain embodiments report methods and compositions for diagnosing and/or treating a subject having a BRCA1-related cancer or sporadic cancer. Some embodiments disclosing treatment can include, but are not limited to, modulation of BRCA1. Certain embodiments report increasing BRCA1 protein stability in a subject having BRCA1-associated cancer.

Some embodiments of the present invention report methods for treating cancer in a subject, including, but not limited to, administering to the subject in need thereof, a therapeutically effective amount of an Akt activator, or Akt inhibitor or a pharmaceutically acceptable salt thereof. Whether an Akt activator or inhibitor can be administered depends in part on the type of cancer (e.g. hereditary or spontaneously appearing cancer) and BRCA1 protein-associated with the subject. In humans, there are three genes in the “Akt family”: Akt1, Akt2, and Akt3. These genes code for enzymes that are members of the serine/threonine-specific protein kinase family. It is contemplated that Akt activation or inhibition in a human may concern one or all of the members of the Akt family.

Other embodiments of the present invention report compositions for treating cancer in a subject and/or compositions for diagnosing cancer in a subject. In accordance with these embodiments, a BRCA1-associated cancer may be diagnosed in a subject. In other embodiments, a subject identified as having a BRCA1-associated cancer may be treated with compositions disclosed herein, for example, an Akt activator and optionally, at least one of a proteosome inhibitor or protease inhibitor. In other embodiments, a subject having, for example, a sporadic cancer may be treated first, with a BRCA1 destabilizer, and then treated with a PARP inhibitor. In other embodiments, a subject having destabilized BRCA1 and a non-sporadic cancer (e.g. hereditary cancer) may be treated with a BRCA1 stabilizer such as Akt activator and optionally, one or more of a proteosome inhibitor and a protease inhibitor.

Yet other embodiments of the present invention disclose kits for using compositions disclosed herein. Some kits report diagnosing a cancer subject having a destabilized BRCA1 protein pool. Some embodiments disclose kits having an anti-BRCA1 antibody. For example, certain kits report antibodies or antibody fragments that associate with phosphorylated serine 694 or phosphorylated threonine 509.

### BRIEF DESCRIPTION OF THE DRAWINGS

While multiple embodiments are disclosed, still other embodiments of the present invention will become apparent to those skilled in the art from the following detailed description, which illustrates and describes embodiments of the invention. Accordingly, the drawings and detailed description are to be regarded as illustrative in nature and not restrictive.

FIGS. 1A-1C represents a Western blots (A and B) and a histogram plot (C) of stabilization of BRCA 1 protein expression using various concentrations of an Akt activator.

FIGS. 2A-2C illustrate blots (A), an exemplary histogram comparison (B), and graphs representing cell cycle progression (C) illustrating accumulation of BRCA1 protein.

FIGS. 3A-3C illustrate blot analyses of PI3-Kinase/Akt signaling regulation of BRCA1 protein levels.

FIGS. 4A-4D represents blots illustrating Akt phosphorylation of BRCA1.

FIGS. 5A-5C represents blots (A and B) and graph (C) illustrating Akt phosphorylation of BRCA1 at positions 5694 and T509.

FIGS. 6A-6B represent blots illustrating increases in BRCA1 protein levels.

FIGS. 7A-7I represents photo illustrations of Akt activation and promotion of nuclear localization of BRCA1.

FIGS. 8A-8B illustrates a graph (A) and blot (B) of co-expression of activated Akt and BRCA1.

FIG. 9 illustrates a blot representing an anti-phosphorylated serine 694 polyclonal antibody of BRCA1.

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some cases, well-known methods or components have not been included in the description.

Embodiments of the present invention generally relate to methods, compositions and uses for analysis and modulations of BRCA1. In some embodiments, methods, compositions and uses for diagnosis and treatment of cancer in a subject are disclosed. Certain embodiments report methods and compositions for diagnosing and/or treating a subject having a BRCA1-related cancer or sporadic cancer. Some embodiments disclose treatments that can include, but are not limited to, modulation of BRCA1 by for example, stabilization of BRCA1 by increasing phosphorylation of serine or threonine residues of BRCA1.

Embodiments of the present invention can provide for methods of diagnosis and treatment of cancers based on modulation of BRCA1. In one aspect, methods are provided for stabilizing BRCA1 in hereditary cancers through the administration of Akt activators alone or in combination with protease inhibitors, proteosome inhibitors or a combination of the two.

In another aspect, methods are provided for destabilization of BRCA1 in some sporadic cancers. In accordance with this aspect, destabilization of BRCA1 may be used in order to transform them into BRCA1-deficient or modified sporadic cancers. In certain embodiments, destabilizing BRCA1 in some sporadic cancers, for example, through administration of Akt inhibitors to a subject having a sporadic cancer, may render such cancer cells susceptible to BRCA1 targeted therapies. Therapies for treating these subjects can include, but are not limited to, PARP inhibitors.

Other embodiments provide for a diagnostic test to determine whether a particular tumor contains a stable BRCA1 protein. Analysis of stability of BRCA1 protein can allow a health professional to select appropriate therapies for treatment of the tumor in a subject. A subject found to have unstable BRCA1 proteins may be a candidate for therapy with Akt activators. Akt activators may be used alone or in combination with one or more of protease inhibitors and proteosome inhibitors. Subjects with stable BRCA1 may be a candidate for therapy with one or more of Akt inhibitors and PARP inhibitors (e.g. a subject with a sporadic cancer).

### Akt Family

Akt possesses a protein domain known as a PH domain, or Pleckstrin Homology domain, the protein in which it was first discovered. This domain binds to phosphoinositides with high affinity. In the case of the PH domain of Akt, it binds either phosphatidylinositol (3,4,5)-trisphosphate (PtdIns(3,4,5)P₃ aka PIP₃) or phosphatidylinositol (3,4)-bisphosphate (PtdIns(3,4)P₂ or PI(3,4)P₂). Because of this feature, this is useful for control of cellular signaling because the di-phosphorylated phosphoinositide PtdIns(4,5)P₂ is phosphorylated by the family of enzymes, PI 3-kinases (phosphoinositide 3-kinase or PI3K), upon receipt of chemical messengers which instruct the cell to commence the growth process. In one example, PI 3-kinases may be activated by a G protein coupled receptor or receptor tyrosine kinase such as the insulin receptor. In this exemplary method of control, once activated, PI 3-kinases phosphorylates PtdIns(4,5)P₂ to form PtdIns(3,4,5)P₃.

### DEFINITIONS

As used herein, “a” or “an” can mean one or more than one of an item.

As used herein, vessel can include, but is not limited to, test tube, mini- or micro-fuge tube, channel, vial, microtiter plate or container.

As used herein the specification, “subject” or “subjects” may include but are not limited mammals such as humans or mammals, domesticated or wild, for example dogs, cats, other household pets (e.g. hamster, guinea pig, mouse, rat), ferrets, rabbits, pigs, horses, cattle, prairie dogs, or zoo animals.

As used herein, “about” can mean plus or minus ten percent.

As used herein, “antibody” can refer to a full-length (e.g., naturally occurring or formed by normal immunoglobulin gene fragment recombinatorial processes) immunoglobulin molecule (e.g., an IgG antibody) or an immunologically active (e.g. binding) portion of an immunoglobulin molecule, like an antibody fragment. The term “antibody” also includes “humanized” antibodies and even fully human antibodies that can be produced by any means known in the art. This term also includes monoclonal antibodies, polyclonal antibodies, multivalent antibodies, multispecific antibodies (e.g., bispecific antibodies).

As used herein, “polyclonal antibodies” can be generated in an immunogenic response to a protein having many epitopes. A composition (e.g., serum) of polyclonal antibodies can thus include a variety of different antibodies directed to the same and to different epitopes within a protein. Methods for producing polyclonal antibodies are known in the art.

As used herein, “antipeptide antibodies” (also known as “monospecific antibodies”) can be generated in a humoral response to a short (e.g 5 to 20 amino acids) immunogenic polypeptide that corresponds to a few (preferably one) isolated epitopes of the protein from which it is derived. A plurality of antipeptide antibodies includes a variety of different antibodies directed to a specific portion of the protein, e.g., to an amino acid sequence that contains at least one, preferably only one, epitope. Methods for producing antipeptide antibodies are known in the art.

As used herein, “monoclonal antibody” can be a specific antibody that recognizes a single specific epitope of an immunogenic protein. In a plurality of a monoclonal antibody, each antibody molecule is identical to the others in the plurality. In certain embodiments, in order to isolate a monoclonal antibody, a clonal cell line that expresses, displays and/or secretes a particular monoclonal antibody is first identified; this clonal cell line can be used in one method of producing the antibodies of the present invention. Methods for preparation of clonal cell lines and of monoclonal antibodies expressed are known in the art.

As used herein, “naked antibody” can be an intact antibody molecule that contains no further modifications such as conjugation with a toxin, or with a chelate for binding to a radionuclide. The Fc portion of the naked antibody can provide effector functions, such as complement fixation and ADCC (antibody dependent cell cytotoxicity), which set mechanisms into action that may result in cell lysis.

As used herein, “antibody fragment” can be a portion of an intact antibody such as F(ab′)a, F(ab)₂, Fab′, Fab, Fv, sFv and the like. Regardless of structure, an antibody fragment binds with the same antigen that is recognized by the full-length antibody. The term “antibody fragment” can also include any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

As used herein, antibody fragments produced by limited proteolysis of wildtype antibodies can be called proteolytic antibody fragments. These include, but are not limited to, the following: “F(ab′)2 fragments” that are released from an antibody by limited exposure of the antibody to a proteolytic enzyme, e.g., pepsin or ficin. An F(ab′)2 fragment comprises two “arms,” each of which comprises a variable region that is directed to and specifically binds a common antigen. The two Fab′ molecules are joined by interchain disulfide bonds in the hinge regions of the heavy chains; the Fab′ molecules may be directed toward the same (bivalent) or different (bispecific) epitopes.

As used herein, “Fab′-SH fragments” can be produced from F(ab′)2 fragments, which are held together by disulfide bond(s) between the H chains in an F(ab′)2 fragment. Treatment with a mild reducing agent such as, by way of non-limiting example, beta-mercaptoethylamine, breaks the disulfide bond(s), and two Fab′ fragments are released from one F(ab′)2 fragment. Fab′-SH fragments are monovalent and monospecific.

As used herein, “Fab fragments” (e.g., an antibody fragment that contains the antigen-binding domain and comprises a light chain and part of a heavy chain bridged by a disulfide bond) can be produced by papain digestion of intact antibodies. One convenient method is to use papain immobilized on a resin so that the enzyme can be easily removed and the digestion terminated. Fab fragments do not have the disulfide bond(s) between the H chains present in an F(ab′)2 fragment.

As used herein, “single-chain antibodies” can be one type of antibody fragment. The term single chain antibody is often abbreviated as “scFv” or “sFv.” These antibody fragments are produced using molecular genetics and recombinant DNA technology. A single-chain antibody consists of a polypeptide chain that comprises both a VH and a VL domains which interact to form an antigen-binding site. The VH and VL domains are usually linked by a peptide of 10 to 25 amino acid residues.

As used herein, “BARDI” can mean a gene encoding a protein, BARDI, which interacts with the N-terminal region of BRCA1 protein. The BARDI/BRCAI protein interaction can be disrupted by tumorigenic amino acid substitutions in BRCA1.

As used herein, “modulation” can mean a change (e.g. increase or decrease) in the level or magnitude of an activity or process. Modulation may be assayed by determining any parameter that indirectly or directly affects a change in a protein, such as truncation, any change in post-translational modification and/or phosphorylation.

As used herein, “PARP” can refer to a protein involved in a number of cellular processes (e.g. Poly (ADP-ribose) polymerase (PARP)).

### Methods of Treatment

In one embodiment, methods for treating cancer in a subject by administering an activator of Akt are provided. In a certain embodiments, types of cancer may include, but are not limited to, breast cancer ovarian cancer or prostate cancer. In some embodiments, an Akt activator can phosphorylate BRCA1 protein of a subject. In other embodiments, an Akt activator can phosphorylate BRCA1 protein of a subject on one or more specific amino acids of BRCA1 protein. In accordance with these embodiments, phosphorylation of BRCA1 by Akt can reduce or prevent proteosomal-mediated degradation and/or promotes cell survival after DNA damage.

Activators of Akt can include, but are not limited to, any molecule that directly or indirectly activates the biological activities of Akt. For example, such activation may include activation of the upstream PI3K-Akt signaling pathway. In one embodiment, Akt activation can induce Akt-dependent phosphorylation of serine 694 and/or threonine 509 of BRCA1, which can stabilize BRCA1 levels. In another embodiment, Akt can be activated by estrogen which activates the PI3K-Akt pathway. In another embodiment, Akt can be activated by IGF-1, which activates the PI3K-Akt pathway. In another embodiment, an Akt activators can be a polypeptides or peptides that modulate Akt activities, but do not directly interact with Akt. For example, activators of Akt activity may be genetically modified Akt molecules which are then artificially activated. Akt activators may include, but are not limited to, estrogen, IGF-1, calcium/calmodulin, insulin, PtdIns-3,4,-P₂, and Ro 31-8220. In certain embodiments, use of modified Akt molecules may be delivered to a subject in need of such a treatment by expression vectors, liposomes, adenoviruses, or any non-viral methods known to one skilled in the art including calcium phosphate precipitation.

In other embodiments, the method for treating cancer in a subject may include, administrating at least one of a protease inhibitor and a proteasome inhibitor. A protease inhibitor may include, but are not limited to, AEBSF, Amastatin-HCL, (ε)-Aminocaproic acid, α1-Antichymotypsin from human plasma, Antipain-HCL, Antithrombin III from human plasma, α1-ntitrypsin from human plasma, α1-proteinase inhibitor, APMSF-HCL, Aprotinin, Arphamenine A, Arphamenine B, Benzamidine-HCL, Bestatin-HCL, CA-074, CA-074-Me, Calpain Inhibitor I, Calpain Inhibitor II, DFP, E-64, EGTA, Elastinal, Leuhistin, Pepstatin A, Phebestin, PMSF, TLCK, and TPCK. The proteasome inhibitors may include, but are not limited to, MG132 and Bortezomib. Proteasome inhibition can reduce or prevent degradation of BRCA1, and may increase stabilization and accumulation of BRCA1 and BARD1. For example, a protease inhibitor and/or a proteasome inhibitor may be administered along with or after administration of an Akt activator.

In yet another embodiment, method of treating cancer may include, administration of an Akt inhibitor. This method can be useful for treating subjects with sporadic cancers. For example, administering an Akt inhibitor may provide for transforming a cancer or subject having a sporadic cancer into a BRCA1-deficient cancers, which have a known mechanism for targeting a therapeutic. In some embodiments, sporadic cancers include, but are not limited to, sporadic breast cancer (non-hereditary), ovarian cancer, and prostate cancer. In such treatment regimes, destabilizing BRCA1, which may occur through the administration of Akt inhibitors, may render such sporadic cancer cells susceptible to BRCA1-deficient cancer-targeted therapies (e.g. PARP inhibitors).

Akt inhibitors may include, but are not limited to, LY 294,002, KP372-1FPA-124, Akt Inhibitor II, Akt Inhibitor III, Akt Inhibitor IV, Akt Inhibitor X, Akt Inhibitor unconjugated, 5-(2-Benzothiazolyl)-3-ethyl-2-[2-(methylphenylamino) ethenyl]-1-phenyl-1H-benzimidazolium iodide, and Triciribine.

PARP inhibitors may include, but are not limited to, 3-amino-benzamide, 8-hydroxy-2-methylquinazolin-4-(3H)-one (NU1025), and AG14361.

In some embodiments, Akt inhibitors may include any molecule that directly or indirectly counteract, reduce, antagonize or inhibit Akt biological activities. In one embodiment, an Akt inhibitor may compete or block binding of Akt to its ligands. In another embodiment, Akt inhibitors may directly interact with Akt. In other embodiments, Akt inhibitors may be antibodies or antibody fragments that bind to Akt and reduce or neutralize at least one biological activity of Akt. In another embodiment, Akt inhibitors may be one or more polypeptides or peptides that modulate Akt activities but do not directly interact with Akt. For example, Akt inhibitors can be mutated Akt molecules, such as dominant-negative mutants derived from a wild-type Akt by terminal truncations or amino acid substitutions. Such mutated Akts may retain binding ability to the signaling molecules of Akt but lose ability of triggering the downstream signaling transduction of Akt. Therefore, the mutated Akt molecules can compete with the wild-type Akt and thus block the activities of the wild-type Akt. Standard mutagenesis and molecular cloning techniques known in the art may be used to perform terminal truncation and/or amino acid substitution. Mutated Akt molecules can be administered into target cells by standard delivery means known in the art, such as, expression vectors, liposomes, adenoviruses, and any methods known to one skilled in the art including calcium phosphate precipitation.

Akt inhibitors may also interact with and regulate upstream activity, including regulation of the PI3K-Akt signaling pathway. Accordingly, any molecules capable of regulating this pathway may be used as an Akt inhibitor. In one example, an Akt inhibitor may be LY 294,002. Any of the Akt inhibitors contemplated herein may be delivered in expression vectors, liposomes, adenoviruses, and any methods known to one skilled in the art including calcium phosphate precipitation.

### Some Methods of Diagnosis

In another aspect, methods to detect whether or not BRCA1 protein of a subject is in a stable form are reported. Detection of low levels of stabilized BRCA1 coupled with normal or high levels of BRCA1 mRNA in tissue can be indicative of a destabilized form of BRCA1. Subjects of this type may be appropriate candidates for treatment with Akt activators alone or in combination with one or more of protease inhibitors and proteosome inhibitors in order to increase stabilized form of BRCA1.

In other embodiments, detection of high levels of BRCA1 in a subject with sporadic cancer of unknown mechanisms can indicate the subject may be a candidate for treatment with Akt activity inhibitors alone or in combination with PARP inhibitors.

Some methods for detection of BRCA1 levels in a tissue sample of a subject may be through any methods known to those skilled in the art, including, for example, ELISA, western blot analysis, or immunohistochemistry (IHC), etc. mRNA levels can be detected by any methods known to those skilled in the art, including a reverse-transcriptase polymerase chain reaction (RT-PCR). Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

In one embodiment, a BRCA1 phosphospecific antibody detecting phosphothreonine 509 and/or phosphoserine 694 may be generated and used to detect phosphorylated BRCA1 proteins in a sample from a subject having cancer. A low level (compared to a control sample from a control subject) or a complete absence of any BRCA1 detection in combination with high levels of mRNA can be useful in identifying subjects who are good candidates for treatment with Akt activators alone or in combination with protease inhibitors, proteosome inhibitors or a combination of both in order to promote a stabilized form of BRCA1.

Alternatively, detection of high-levels of phosphorylated BRCA1 proteins in a subject having a sporadic cancer can be indicative that the subject may be a good candidate for treatment with Akt inhibitors alone or in combination with PARP inhibitors.

Any antibody specific for a phosphorylated form of BRCA1 (e.g. phosphorylated serine 694 or phosphorylated threonine 509 of BRCA1) may be used in some embodiments of the present invention. Means for preparing and characterizing antibodies are well known in the art (See, e.g., Antibodies: A Laboratory Manual, Cold Spring Harbor Laboratory, 1988; incorporated herein by reference). Methods for generating polyclonal antibodies are well known in the art. Briefly, a polyclonal antibody is prepared by immunizing an animal with an immunogenic composition and collecting antisera from that immunized animal. A wide range of animal species may be used for the production of antisera. Typically the animal used for production of anti-antisera is a rabbit, a mouse, a rat, a hamster, a guinea pig, a chicken or a goat. Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells.

In some embodiments, level of binding of a phosphospecific antibody to BRCA1 protein in a sample can be assessed using a rapid screening technique. Certain examples of these techniques include, but are not limited to, IHC, western blot analysis, Elisa, immunoprecipitation, radioimmunoassay, mass spectroscopy, gas chromatography-mass spectroscopy, two-dimensional electrophoresis and staining with organic dyes, metal chelates, fluorescent dyes, complexing with silver, or pre-labeling with fluorophores, as well as any future technology capable of ascertaining the level of phosphospecific antibody binding to a sample.

In still further embodiments, immunodetection methods for binding, purifying, removing, quantifying or otherwise generally detecting biological components may be used. Antibodies prepared in accordance with embodiments of the present invention may be employed to detect the encoded proteins or peptides. Various useful immunodetection methods have been described in the scientific literature, such as, e.g., Nakamura et al. (1987).

In some embodiments regarding antigen detection, a biological sample analyzed may be any sample and can include, but are not limited to, a tissue section or specimen, a homogenized tissue extract, an isolated cell, a cell membrane preparation, separated or purified forms of any of the above protein-containing compositions or even any biological fluid. Various embodiments include bone marrow aspirate, bone marrow biopsy, lymph node aspirate, lymph node biopsy, spleen tissue, fine needle aspirate, skin biopsy or organ tissue biopsy. Other embodiments include samples where the body fluid is peripheral blood, lymph fluid, ascites, serous fluid, pleural effusion, sputum, cerebrospinal fluid, lacrimal fluid, stool or urine.

Antibodies may be used in conjunction with both fresh-frozen and formalin-fixed, paraffin-embedded tissue blocks prepared by IHC. Any IHC method known in the art may be used such as those described in Diagnostic Immunopathology, 2nd edition. edited by, Robert B. Colvin, Atul K. Bhan and Robert T. McCluskey. Raven Press, New York, 1995, (incorporated herein by reference).

Suitable conditions for binding an antibody can mean that the incubation is at a temperature and for a period of time sufficient to allow effective binding. Incubation steps are typically from about 1 to 2 to 4 hours, at temperatures preferably on the order of 25° to 27° C., or may be overnight at about 4° C. or so. Antibodies contemplated, can be used to quantify and localize the expression of encoded marker proteins. The antibody, for example, can be labeled by any one of a variety of methods and used to visualize the localized concentration of the cells producing the encoded protein.

In vivo methods of imaging a cancerous condition are reported, for example, using the above-described antibodies. This method can involve administering to a subject an imaging-effective amount of a detectably-labeled disease-specific monoclonal antibody or fragment thereof and a pharmaceutically effective carrier and detecting the binding of the labeled antibody to the diseased tissue. The term “in vivo imaging” can refer to any method which permits the detection of a labeled antibody of the present invention or fragment thereof that specifically binds to a diseased tissue located in the subject's body. An “imaging effective amount” can mean that the amount of the detectably-labeled antibody, or fragment thereof, administered is sufficient to enable detection of binding of the monoclonal antibody or fragment thereof to the diseased tissue.

A factor to consider in selecting a radionuclide for in vivo diagnosis can be that the half-life of a nuclide be long enough so that it is still detectable at the time of maximum uptake by the target, but short enough so that deleterious radiation upon the host, as well as background, is minimized. In one example, a radionuclide used for in vivo imaging can lack a particulate emission, but produce a large number of photons in a 140-2000 keV range, which may be readily detected by conventional gamma cameras. A radionuclide may be bound to an antibody either directly or indirectly by using an intermediary functional group. Intermediary functional groups which are often used to bind radioisotopes which exist as metallic ions to antibody are diethylenetriaminepentaacetic acid (DTPA) and ethylene diaminetetraacetic acid (EDTA). Examples of metallic ions suitable for use in this invention are ^{99m}Tc, ¹²³I, ¹³¹I, ¹¹¹In, ¹³¹I, ⁹⁷Ru, ⁶⁷Cu, ⁶⁷Ga, ¹²⁵I, ⁶⁸Ga, ⁷²As, ⁸⁹Zr, and ²⁰¹Tl.

Administration of the labeled antibody may be local or systemic and accomplished intravenously, intraarterially, via the spinal fluid or the like. Administration may also be intradermal or intracavitary, depending upon the body site under examination. After a sufficient time has lapsed for the monoclonal antibody or fragment thereof to bind with the diseased tissue, for example 30 minutes to 48 hours, the area of the subject under investigation is examined by routine imaging techniques such as MRI, SPECT, planar scintillation imaging and emerging imaging techniques, as well. An exact protocol can vary depending upon factors specific to the subject, as noted above, and depending upon the body site under examination, method of administration and type of label used; the determination of specific procedures would be routine to the skilled artisan. Distribution of the bound radioactive isotope and its increase or decrease with time is then monitored and recorded. By comparing the results with data obtained from studies of clinically normal individuals, the presence and extent of the diseased tissue may be determined.

### Aptamers

In certain embodiments, a nucleic acid sequence or ligand of use may be an aptamer, for example to bind to phosphorylated BRCA1 protein. Methods of constructing and determining the binding characteristics of aptamers are well known in the art. For example, such techniques are described in U.S. Pat. Nos. 5,582,981, 5,595,877 and 5,637,459, each incorporated herein by reference. In some embodiments, an aptamer may be generated to recognize and bind to a phosphoserine or phosphothreonine of BRCA1 (e.g. a nucleic acid ligand of part or all of SEQ ID NO:10).

Aptamers may be prepared by any known method, including synthetic, recombinant, and purification methods, and may be used alone or in combination with other ligands specific for the same target. In general, a minimum of approximately 3 nucleotides, preferably at least 5 nucleotides, can be generated that effect specific binding.

Aptamers may be extended with flanking regions and otherwise derivatized, for example, flanking by primer-binding sequences, facilitating the amplification of the aptamers by PCR or other amplification techniques.

Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S, Not all linkages in an oligomer need to be identical.

Methods for preparation and screening of aptamers that bind to particular targets of interest are well known, for example U.S. Pat. No. 5,475,096 and U.S. Pat. No. 5,270,163, each incorporated by reference.

### Methods of Disease Tissue Detection, Diagnosis and Imaging Protein Based In Vitro Diagnosis

Embodiments of the present invention contemplates use of nucleic acid ligands, including BRCA1 derived binding peptides, BRCA1 fusion proteins, BRCA1 antibodies or fragments, bi-specific antibodies and antibody fragments, to screen biological samples in vitro and/or in vivo for the presence of BRCA1 molecules. In exemplary immunoassays, the BRCA1 antibody, fusion protein, or fragment thereof may be utilized in liquid phase or bound to a solid-phase carrier, as described below. In certain embodiments, for example, in vivo administration, the BRCA1 antibody or fragment thereof is humanized. In other embodiments, for example, the BRCA1 antibody or fragment thereof is fully human. The skilled artisan will realize that a wide variety of techniques are known for determining levels of expression of a particular gene and any such known method, such as immunoassay, RT-PCR, mRNA purification and/or cDNA preparation followed by hybridization to a gene expression assay chip may be utilized to determine levels of BRCA1 expression or modification or stability in individual subjects and/or tissues.

One example of a screening method for determining whether a biological sample contains the BRCA1 protein is radioimmunoassay (RIA). For example, in one form of RIA, the substance under test is mixed with BRCA1 Ab in the presence of radiolabeled BRCA1 antigen. In this method, the concentration of the test substance will be inversely proportional to the amount of labeled BRCA1 antigen bound to the Ab and directly related to the amount of free, labeled BRCA1 antigen. Other suitable screening methods will be readily apparent to those of skill in the art.

Alternatively, in vitro assays may be performed in which a BRCA1 ligand, anti-BRCA1 antibody, fusion protein, or fragment thereof is bound to a solid-phase carrier. For example, Abs can be attached to a polymer, such as aminodextran, in order to link the Ab to an insoluble support such as a polymer-coated bead, a plate or a tube.

The presence of the BRCA1 protein or antigen in a biological sample may be determined using an enzyme-linked immunosorbent assay (ELISA). In the direct competitive ELISA, a pure or semipure antigen preparation is bound to a solid support that is insoluble in the fluid or cellular extract being tested and a quantity of detectably labeled soluble antibody, antibody fragment or BRCA1 ligand is added to permit detection and/or quantitation of the binary complex formed between solid-phase antigen and labeled BRCA1 binding molecule.

### Nucleic Acid Based In Vitro Diagnosis

In some embodiments, nucleic acids may be analyzed to determine levels of BRCA1 expression, for example, using nucleic acid amplification methods. Nucleic acid sequences (e.g. mRNA and/or cDNA) to be used as a template for amplification may be isolated from cells contained in a biological sample, according to standard methodologies. A nucleic acid may be fractionated or whole cell RNA. Where RNA is used, it may be desired to convert the RNA to a complementary cDNA. In one embodiment, the RNA is whole cell RNA and can be used directly as the template for amplification.

In one example, the determination of BRCA1 expression is performed by amplifying (e.g. by PCR) the BRCA1 mRNA or cDNA sequences and detecting and/or quantifying an amplification product by any methods known in the art, including but not limited to TaqMan assay (Applied Biosystems, Foster City, Calif.), agarose or polyacrylamide gel electrophoresis and ethidium bromide staining, hybridization to a microarray comprising a BRCA1 specific probe, Northern blotting, dot-blotting, slot-blotting, etc.

Various forms of amplification are well known in the art and any such known method may be used. Generally, amplification involves the use of one or more primers that hybridize selectively or specifically to a target nucleic acid sequence to be amplified.

One embodiment of the invention may comprise obtaining a suitable sample from an individual and detecting a BRCA1 messenger RNA. Once the tissue sample is obtained the sample may be prepared for isolation of the nucleic acids by standard techniques (eg, cell isolation, digestion of outer membranes, Oligo dT isolation of mRNA etc.) The isolation of the mRNA may also be performed using kits known to the art (Pierce, AP Biotech, etc). A reverse transcriptase PCR amplification procedure may be performed in order to quantify an amount of mRNA amplified. Methods of reverse transcribing RNA into cDNA are well known and described in Sambrook et al., 1989. Alternative methods for reverse transcription utilize thermostable DNA polymerases.

The above-described in vitro and in situ detection methods may be used to assist in the diagnosis or staging of a pathological condition. For example, such methods can be used to detect tumors that express the BRCA1 antigen, such as metastatic cancer.

### In Vivo Diagnosis

BRCA1 ligands and/or antibodies are of use for in vivo diagnosis. Methods of diagnostic imaging with labeled peptides or Abs are well-known. For example, in the technique of immunoscintigraphy, BRCA1 ligands or antibodies can be labeled with a gamma-emitting radioisotope and introduced into a subject. For diagnostic imaging, radioisotopes may be bound to the BRCA1 ligand or antibody either directly, or indirectly by using an intermediary functional group. Useful intermediary functional groups can include, but are not limited to, chelators such as ethylenediaminetetraacetic acid and diethylenetriaminepentaacetic acid. For example, see Shih et al., supra, and U.S. Pat. No. 5,057,313. Examples of radioisotopes that can be bound to BRCA1 antibody and are appropriate for diagnostic imaging include ^{99m}Tc and ¹¹¹In.

BRCA1 ligands, aptamers, antibodies, fusion proteins, and fragments thereof also can be labeled with paramagnetic ions and a variety of radiological contrast agents for purposes of in vivo diagnosis. Contrast agents that are particularly useful for magnetic resonance imaging comprise gadolinium, manganese, dysprosium, lanthanum, or iron ions. Additional agents include chromium, copper, cobalt, nickel, rhenium, europium, terbium, holmium, or neodymium. BRCA1 ligands, antibodies and fragments thereof can also be conjugated to ultrasound contrast/enhancing agents.

In one embodiment, a bispecific antibody can be conjugated to a contrast agent. For example, a bispecific antibody may comprise more than one image-enhancing agent for use in ultrasound imaging. In another embodiment, a contrast agent is a liposome. Preferably, the liposome comprises a bivalent DTPA-peptide covalently attached to the outside surface of the liposome.

### Imaging Agents and Radioisotopes

In certain embodiments, the claimed peptides or proteins may be attached to imaging agents of use for imaging and diagnosis of various diseased organs, tissues or cell types. Many appropriate imaging agents are known in the art, as are methods for their attachment to proteins or peptides (see, e.g., U.S. Pat. Nos. 5,021,236 and 4,472,509, both incorporated herein by reference).

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶², copper⁶⁴, copper⁶⁷, ¹⁵²Eu, fluorine¹⁸, gallium⁶⁷, gallium⁶⁸, ³hydrogen, iodine¹²³, iodine¹²⁴, iodine¹²⁵, iodine¹³¹, indium¹¹¹, ⁵²iron, ⁵⁹iron, ³²phosphorus, ³³phosphorus, rhenium¹⁸⁶, rhenium¹⁸⁸, Sc⁴⁷, ⁷⁵selenium, silver¹¹¹, ³⁵sulphur, technicium^{94m} technicium^{99m} yttrium⁸⁶ and yttrium⁹⁰. ¹²⁵I is often being preferred for use in certain embodiments, and technicium^{99m} and indium¹¹¹ are also often preferred due to their low energy and suitability for long range detection.

Radioactively labeled proteins or peptides may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides may be labeled with technetium-^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups which are often used to bind radioisotopes which exist as metallic ions to peptides include diethylenetriaminepentaacetic acid (DTPA), DOTA, NOTA, porphyrin chelators and ethylene diaminetetracetic acid (EDTA). Also contemplated for use are fluorescent labels, including rhodamine, fluorescein isothiocyanate and renographin.

In certain embodiments, proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference. These fluorescent labels are preferred for in vitro uses, but may also be of utility in in vivo applications, particularly endoscopic or intravascular detection procedures.

In alternative embodiments, BRCA1 ligands, antibodies, or other proteins or peptides may be tagged with a fluorescent marker. Photodetectable labels are known in the art and are contemplated for some embodiments of the present invention.

Chemiluminescent labeling compounds of use may include luminol, isoluminol, an aromatic acridinium ester, an imidazole, an acridinium salt and an oxalate ester, or a bioluminescent compound such as luciferin, luciferase and aequorin. Diagnostic immunoconjugates may be used, for example, in intraoperative, endoscopic, or intravascular tumor or disease diagnosis.

In various embodiments, labels of use may comprise metal nanoparticles. Methods of preparing nanoparticles are known in the art. (See e.g., U.S. Pat. Nos. 6,054,495; 6,127,120; 6,149,868; Lee and Meisel, J. Phys. Chem. 86:3391-3395, 1982.)

### Cross-Linkers

In some embodiments, proteins or peptides may be labeled using various cross-linking reagents known in the art, such as homo-bifunctional, hetero-bifunctional and/or photoactivatable cross-linking reagents. Such reagents may be modified to attach various types of labels, such as fluorescent labels.

### Vectors for Cloning, Gene Transfer and Expression

In certain embodiments, expression vectors may be employed to express peptides or proteins, such as fusion proteins, which can then be purified and used. In other embodiments, the expression vectors may be used, for example, in gene therapy. Expression requires that appropriate signals be provided in the vectors, and which include various regulatory elements, such as enhancers/promoters from either viral or mammalian sources that drive expression of the genes of interest in host cells. Elements designed to optimize messenger RNA stability and translatability in host cells are known and contemplated herein.

### Regulatory Elements

The terms “expression construct” or “expression vector” can include any type of genetic construct containing a nucleic acid coding for a gene product in which part or all of the nucleic acid coding sequence is capable of being transcribed.

In various embodiments, the human cytomegalovirus (CMV) immediate early gene promoter, the SV40 early promoter, the Rous sarcoma virus long terminal repeat, rat insulin promoter, and glyceraldehyde-3-phosphate dehydrogenase promoter can be used to obtain high-level expression of the coding sequence of interest. Use of other viral or mammalian cellular or bacterial phage promoters which are well-known in the art to achieve expression of a coding sequence of interest is contemplated as well, provided that the levels of expression are sufficient for a given purpose.

### Selectable Markers

In certain embodiments, cells containing nucleic acid constructs may be identified in vitro or in vivo by including a marker in the expression construct. Such markers would confer an identifiable change to the cell permitting easy identification of cells containing the expression construct. Further examples of selectable markers are well known to one of skill in the art.

### Delivery of Expression Vectors

There are a number of ways in which expression vectors may introduced into cells. In certain embodiments, the expression construct comprises a virus or engineered construct derived from a viral genome. The ability of certain viruses to enter cells via receptor-mediated endocytosis, to integrate into host cell genome, and express viral genes stably and efficiently have made them attractive candidates for the transfer of foreign genes into mammalian cells (for example, Ridgeway, In: Vectors: A Survey of Molecular Cloning Vectors and Their Uses, Rodriguez et al., eds., Stoneham: Butterworth, pp. 467-492, 1988).

DNA viruses used as gene vectors can include, but are not limited to papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

Other gene transfer vectors may be constructed from retroviruses. The retroviruses can be a group of single-stranded RNA viruses characterized by an ability to convert their RNA to double-stranded DNA in infected cells by a process of reverse-transcription (Coffin, In: Virology, Fields et al., eds., Raven Press, New York, pp. 1437-1500, 1990).

Other viral vectors may be employed as expression constructs. Vectors derived from viruses such as vaccinia virus (see for example, Ridgeway, 1988; Baichwal and Sugden, 1986; Coupar et al., Gene, 68:1-10, 1988), adeno-associated virus (AAV) (Ridgeway, 1988; Baichwal and Sugden, 1986; Hermonat and Muzycska, Proc. Natl. Acad. Sci. USA, 81:6466-6470, 1984), and herpes viruses may be employed.

### Pharmaceutical Compositions

In some embodiments, a BRCA1 ligand, aptamer or antibody or other composition reported and/or one or more other therapeutic agents may be administered to a subject, such as a subject with cancer, for diagnostic or therapeutic purposes. Such agents may be administered in the form of pharmaceutical compositions. Generally, this will entail preparing compositions that are essentially free of impurities that could be harmful to humans or animals.

One generally will employ appropriate salts and buffers to render therapeutic agents stable and allow for uptake by target cells. Aqueous compositions may comprise an effective amount of a BRCA1 binding protein or peptide, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the BRCA1 ligands disclosed herein, its use in therapeutic compositions is contemplated. Supplementary active ingredients also can be incorporated into the compositions.

Methods and compositions claimed herein may include classic pharmaceutical preparations. Administration of these compositions may occur via any common route so long as the target tissue is available via that route. This includes, but are not limited to, oral, nasal, buccal, rectal, vaginal or topical. Alternatively, administration may be by orthotopic, intradermal, subcutaneous, intramuscular, intraperitoneal, intrathecal, intraarterial or intravenous injection. Such compositions normally would be administered as pharmaceutically acceptable compositions.

Pharmaceutical forms suitable for use can include, but are not limited to, sterile aqueous solutions or dispersions and sterile powders for the preparation of sterile solutions or dispersions. For example, in order to be more stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. Prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the compositions can be brought about by the use in the compositions of agents delaying absorption, for example, aluminum monostearate and gelatin.

One skilled in the art would know that a pharmaceutical composition can be administered to a subject by various routes including, for example, orally or parenterally, such as intravenously. In some cases, a BRCA1 ligand may be displayed on the surface of or incorporated into a liposome. Liposomes consist of phospholipids or other lipids, and are generally nontoxic, physiologically acceptable and metabolizable carriers that are relatively simple to make and administer.

In certain embodiments, an effective amount of a therapeutic agent, such as a BRCA1 ligand, must be administered to the subject. An “effective amount” is the amount of the agent that produces a desired effect. An effective amount will depend, for example, on the efficacy of the agent and on the intended effect. An effective amount of a particular agent for a specific purpose can be determined using methods well known to those in the art.

### Therapeutic Agents

Chemotherapeutic Agents

In certain embodiments, chemotherapeutic agents may co-administered with one or more BRCA1 modifying agents, for example, Akt activators or Akt inhibitors. Chemotherapeutic agents may include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts). Some variation in dosage will necessarily occur depending on the condition of the subject being treated. The health professional responsible for administration will, in any event, determine the appropriate dose for the individual subject.

### Hormones

Corticosteroid hormones can increase the effectiveness of other chemotherapy agents, and consequently, they are frequently used in combination treatments. Prednisone and dexamethasone are examples of corticosteroid hormones. Progestins such as hydroxyprogesterone caproate, medroxyprogesterone acetate, and megestrol acetate have been used in cancers of the endometrium and breast. Androgens such as testosterone propionate and fluoxymesterone have also been used in treating breast cancer.

### Immunomodulators

As used herein, the term “immunomodulators” can include, but are not limited to, cytokines, stem cell growth factors, lymphotoxins and hematopoietic factors, such as interleukins, colony stimulating factors, interferons (e.g., interferons-α, -β and -γ) and the stem cell growth factor designated “S1 factor.” Examples of suitable immunomodulator moieties include IL-2, IL-6, IL-10, IL-12, IL-18, IL-21, interferon-gamma, TNF-alpha, and the like. In certain embodiments, immunomodulators may be used in combination with compositions disclosed herein.

Embodiments herein provide for administration of compositions, agents and compounds disclosed herein to a subject in a biologically compatible form suitable for pharmaceutical administration in vivo. Biologically compatible forms can be active agents (e.g. pharmaceutical chemical, protein, peptide, gene, antibody, etc. of some embodiments of the present invention) to be administered. Administration of a therapeutically effective amount of a composition can mean an amount effective, at dosages and for periods of time necessary to achieve the desired result.

In one embodiment, a compound, composition, agent (e.g. a pharmaceutical chemical, protein, peptide, gene, antibody, etc. of the some embodiment of the present invention) may be administered in any manner appropriate for the compound composition, or agent. For example, an agent may be administered to a subject via subcutaneous, intravenous, by oral administration, inhalation, transdermal application, intravaginal application, topical application, intranasal or rectal administration. Depending on the route of administration, an active compound, agent or composition may be coated or carry a material to protect the compound, agent or composition from degradation or contamination etc. by enzymes, acids and other natural conditions that may inactivate the compound.

In some embodiments, a compound may be administered to a subject in an appropriate carrier or diluents, co-administered with enzyme inhibitors or in an appropriate carrier such as liposomes. The term “pharmaceutically acceptable carrier” as used herein can include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

In certain embodiments, therapeutic agents may be formulated within a mixture to include about 0.0001 to 1.0 milligrams, or about 0.001 to 0.1 milligrams, or about 0.1 to 1.0 or even about 1 to 10 grams per dose. Single dose or multiple doses can also be administered on an appropriate schedule for a predetermined condition for example, twice daily, daily, bi-weekly and so on.

In another embodiment, nasal solutions or sprays, aerosols or inhalants may be used to deliver the compound of interest. Additional formulations that are suitable for other modes of administration can include suppositories and pessaries. A rectal pessary or suppository may also be used. In general, for suppositories, traditional binders and carriers may include, for example, polyalkylene glycols or triglycerides; such suppositories may be formed from mixtures containing the active ingredient in the range of 0.5% to 10%, preferably 1% to 2%.

It will be apparent that, for any particular subject, specific dosage regimens may be adjusted over time according to individual need. Doses for administration can be anywhere in the range between about 0.01 mg and about 100 mg per ml of biologic fluid of treated subject. In one embodiment, a range can be between 1 and 100 mg/kg which can be administered daily, every other day, biweekly, monthly, etc. In another embodiment, the range can be between 10 and 75 mg/kg introduced weekly to a subject.

Tablets, troches, pills, capsules, and the like may also contain the following: a binder, as gum tragacanth, acacia, cornstarch, or gelatin; excipients, such as dicalcium phosphate; a disintegrating agent, such as cornstarch, potato starch, alginic acid and the like; a lubricant, such as magnesium stearate; and a sweetening agent, such as sucrose, lactose, or saccharin may be added, or a flavoring agent.

Treatment methods of embodiments of the present invention can be useful in any subject, including any type of mammal and, in certain embodiments, humans.

### Kits

Various embodiments may concern kits containing components suitable for treating or diagnosing diseased tissue in a subject. Exemplary kits may contain at least one BRCA1 directed antibody (e.g. phosphospecific antibody). Optionally, other kit ingredients may include one or more Akt activators, Akt inhibitors, proteosome inhibitors, protease inhibitors, chemotherapeutic agents, bi-specific antibodies or other ingredients as discussed above.

If the composition containing components for administration is not formulated for delivery via the alimentary canal, such as by oral delivery, a device capable of delivering the kit components through some other route may be included. One type of device, for applications such as parenteral delivery, can be a syringe that is used to inject the composition into the body of a subject. Inhalation devices may also be used.

In certain embodiments, kit components may be packaged together or separated into two or more separate containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

### Additional Embodiments

Some embodiments can include a pharmaceutical composition including, a pharmaceutically acceptable amount of at least one Akt activator; and a pharmaceutically acceptable amount of at least one of a protease inhibitor and a proteosome inhibitor or a pharmaceutically acceptable salt thereof. For examples, an Akt activator can be one or more of estrogen, IGF-I, calcium/calmodulin, insulin, PtdIns-3.4,-P2, Ro 31-8220 and a combination thereof. In addition, a pharmaceutical composition can include a protease inhibitor, for example one or more of AEBSF, Amastatin-HCL, (ε)-Aminocaproic acid, α1-Antichymotypsin from human plasma, Antipain-HCL, Antithrombin III from human plasma, α1-ntitrypsin from human plasma, α1-proteinase inhibitor, APMSF-HCL, Aprotinin, Arphamenine A, Arphamenine B, Benzamidine-HCL, Bestatin-HCL, CA-074, CA-074-Me, Calpain Inhibitor I, Calpain Inhibitor II, DFP, E-64, EGTA, Elastinal, Leuhistin, Pepstatin A, Phebestin, PMSF, TLCK, TPCK, and a combination thereof. A pharmaceutical composition may include a proteosome inhibitor, such as, MG132, Bortezomib, any other known proteosome inhibitor or combination thereof.

Other embodiments can include a pharmaceutical composition including a pharmaceutically acceptable amount of at least one Akt inhibitor; and a pharmaceutically acceptable amount of at least one PARP inhibitor or a pharmaceutically acceptable salt thereof. A pharmaceutical composition can include an Akt inhibitor having at least one of LY 294,002, KP372-1FPA-124, Akt Inhibitor II, Akt Inhibitor III, Akt Inhibitor IV, Akt Inhibitor X, Akt Inhibitor unconjugated, 5-(2-Benzothiazolyl)-3-ethyl-2-[2-(methylphenylamino) ethenyl]-1-phenyl-1H-benzimidazolium iodide, Triciribine and a combination thereof. Another pharmaceutical composition can include a PARP inhibitor where the PARP inhibitor includes at least one of 3-amino-benzamide, 8-hydroxy-2-methylquinazolin-4-(3H)-one (NU I 025), AG14361, and a combination thereof.

In another embodiment, a method of treating cancer in a subject can include, identifying a subject having a sporadic cancer, analyzing BRCA1 protein of the subject, administering to the subject having an increased amount of BRCA1 protein compared to a control subject not having a sporadic cancer, a composition having a therapeutically effective amount of an Akt inhibitor, or a pharmaceutically acceptable salt thereof. In accordance with these embodiments, the subject can be further administered a PARP inhibitor. In certain embodiments, a PARP inhibitor can include, but is not limited to, 3-amino-benzamide, 8-hydroxy-2-methylquinazolin-4-(3H)-one (NU I 025), AG14361 or combination thereof.

Some embodiments report treating a subject with cancer, including, but not limited to, introducing to the subject an expression vector encoding an Akt activator, wherein an amount of the Akt activator effective to treat the cancer is expressed in the subject. Other embodiments, can include, for example, administering an expression vector encoding at least one of a protease inhibitor and a proteosome inhibitor alone or along with an expression vector encoding an Akt activator.

Yet further embodiments disclose kits having one or more compositions including, but not limited to, a therapeutically effective amount of an agent selected from an Akt activator or an Akt inhibitor, and optionally, one or more of a protease inhibitor, a proteosome inhibitor, and a PARP; and one or more suitable containers.

Other embodiments include an isolated polynucleotide of TSKRHDphosphoS-DTFPELK (SEQ ID NO:10) associated with a isolated polynucleotide of 30 amino acids or less surrounding phosphorylated threonine 509 of BRCA1. Additionally, other embodiments include a nucleic acid encoding an antibody or fragment thereof, wherein the antibody or fragment thereof binds TSKRHDphosphoS-DTFPELK (SEQ ID NO:10).

Certain embodiments include, methods of stimulating an anti-cancer response in a subject expressing unstable BRCA1, comprising administering to the subject an effective amount of an Akt activator, and optionally one or more of a protease inhibitor or proteosome inhibitor.

A nucleic acid encoding an antibody or fragment thereof, wherein the antibody or fragment thereof binds TSKRHDphosphoS-DTFPELK (SEQ ID NO:10).

### EXAMPLES

The following examples are included to demonstrate preferred embodiments of the invention. It should be appreciated by those of skill in the art that the techniques disclosed in the examples which follow represent techniques discovered to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

### Cell Culture and Chemicals

MCF7 cells were obtained from the American Type Culture Collection (#HTB-22). T47D cells were provided by Baylor College of Medicine. Both lines were normally maintained at 37° C. with 5% CO₂ in DMEM with 1 mM L-glutamine (Gibco) supplemented with 10% Fetalclone serum (HyClone) and 1% non-essential amino acids (Gibco). For steroid depleted conditions (denoted as CSS), cells were cultured in phenol-free DMEM with 1 mM L-glutamine and 25 mM HEPES supplemented with 10% charcoal/dextran treated fetal bovine serum (Gemini Bioproducts). 17-β-estradiol and MG132 were obtained from Sigma-Aldrich. Cycloheximide, U0126, and p38 inhibitor were obtained from Calbiochem. ICI 182780 was obtained from Tocris Biosciences. LY294002 was obtained from Cell Signaling Technology.

### Immunoblot, Immunoprecipitation, and Antibodies

Whole cell lysates were prepared by washing cells twice with PBS and then scraping into ice-cold PBS. Pelleted cells were resuspended in modified RIPA buffer (50 mM Tris base, 150 mM NaCl, 2 mM EDTA, 1% Triton X-100, 0.5% Na-deoxycholate, 0.1% SDS, 50 mM NaF, 5 mM Na₃VO₄, plus Roche protease inhibitor tablets). Lysates were incubated for 30 minutes on ice, clarified by centrifugation, and protein concentration determined by Bradford assay (BioRad). Where indicated, lysates harvested without phosphatase inhibitors were treated with λ-phosphatase (New England Biolabs) according to manufacturer's instructions. 100 μg of total protein was loaded per lane for Tris-glycine SDS-PAGE. Samples were transferred to PVDF membrane (Millipore) for 200 volt-hours at 50 V constant at 16° C. Membranes were blocked in 5% dry non-fat milk (Carnation) dissolved in either PBS or TBS (25 mM Tris pH 8.0, 135 mM NaCl, 2.5 mM KCl). Primary antibody incubations were performed overnight at 4° C. diluted in either 0.5% dry non-fat milk/PBS-Tween-20 0.1% or in 3% BSA (Santa Cruz Biotechnology)/TBS-Tween-20 0.1%. Antibody suppliers are: BRCA1 (Ab-4 and Ab-1, Calbiochem); BARD1 (Bethyl Laboratories); Lamin A/C, phospho-(Ser/Thr) Akt Substrate, phospho-5473 Akt, and Akt (Cell Signaling Technology); Cyclin D1 (Lab Vision Corp.); Tubulin (Chemicon). Secondary antibodies were obtained from Amersham/GE Healthcare. Densitometry was performed on 600 dpi TIFF scans of Western blot films with Quantity One software (BioRad). Density values for BRCA1 and BARD1 in each sample were normalized by the value of the corresponding Lamin A/C density as a control for loading.

For immunoprecipitation, cells were harvested by a similar protocol as described above although cells were lysed in IP lysis buffer (20 mM Tris pH 8, 120 mM NaCl, 1 mM EDTA, 1 mM EGTA, 1% NP-40, 0.25% Na-deoxycholate, 50 mM NaF, 1 mM Na₃VO₄, plus Roche protease inhibitor cocktail tablets). 1 mg of total protein was diluted in IP buffer (10 mM Tris pH 7.4, 50 mM NaCl, 5 mM EDTA, 0.5% CHAPS, 50 mM NaF, 1 mM Na₃VO₄), and 1 μg each of Ab-1 and Ab-4 antibodies was added with Exacta-Cruz immunoprecipitation matrix (Santa Cruz Biotechnology) according to manufacturer's protocols. Control immunoprecipitation was performed with 2 μg of anti-tubulin. SDS-PAGE/WB was performed as described above with the use of Exacta-Cruz secondary antibodies following manufacturer's protocols (Santa Cruz Biotechnology).

### RT-PCR and Cell Cycle Analysis

RNA was isolated from cells using a RNeasy kit (Qiagen) according to manufacturer's instructions. cDNA was synthesized using the SuperScript III First Strand Synthesis System (Invitrogen) with random hexamers. PCR was performed with Platinum PCR SuperMix (Invitrogen) for 25 cycles (pS2 and 36B4) or 27 cycles (BRCA1 and BARD1). Primers are:

(SEQ ID NO: 1)
BRCA1 forward 5′ GAACGGGCTTGGAAGAAAATAATC 3′;
(SEQ ID NO: 2)
reverse 5′ TCAAGGGCAGAAGAGTCAC 3′;
(SEQ ID NO: 3)
BARD1 forward 5′ GCCTGTCGATTATACAGATGATGAAA 3′;
(SEQ ID NO: 4)
reverse 5′ CGCTGCCCAGTGTTCATTACT 3′;
(SEQ ID NO: 5)
pS2 forward 5′ TTCTATCCTAATACCATCGACG 3′;
(SEQ ID NO: 6)
reverse 5′ TTTGAGTAGTCAAAGTCAGAGC 3′;
(SEQ ID NO: 7)
36B4 forward 5′ CTCAACATCTCCCCCTTCTC 3′;
(SEQ ID NO: 8)
reverse 5′ CAAATCCCATATCCTCGTCC 3′.

Samples were run on 1.5% agarose gels, stained with ethidium bromide, and images were captured and analyzed with Quantity One software (BioRad). For cell cycle analysis, cells were cultured as described, trypsinized, and recovered in culture medium. Pelleted cells were washed twice with ice-cold PBS and resuspended in Krishan's stain. Analysis was performed on a Becton Dickinson FC500 with ModFit LT Software (Verity Software House) by the University of Colorado Cancer Center Flow Cytometry Core.

### Recombinant Human Adenovirus

Construction of recombinant human adenovirus expressing wild-type BRCA1 (Ad-BRCA1) or 1853stop truncated BRCA1 (Ad-1853) have been previously described. Recombinant human adenovirus expressing myr-Akt or kd-Akt have been previously described. High titer stocks were generated by infection of HEK-293 packaging cells and CsCl banding, followed by dialysis into viral storage buffer (10 mM Tris pH 7.4, 10 mM histidine, 75 mM NaCl, 1 mM MgCl₂, 0.1 mM EDTA, 0.5% EtOH v/v, 50% glycerol v/v). Viral concentrations were determined by spectrophotometer as previously described.

### GST-BRCA1 Fusion Proteins

Oligonucleotides containing threonine 509 mutated to alanine or glutamic acid were cloned into pBluescript-BRCA1 digested with Bsa1 and EcoN1. Mutations were confirmed by DNA sequencing. PCR products were produced spanning nucleotides 1323-1886 (T509 and A509), 1926-2538 (S694/T696), 3714-4310 (T1246), and 1323-2538 (T/A/E509+S694). These products were cloned into pGEX 4T-1 (GE Healthcare) digested with EcoR1 and Xho1. Production of fusion proteins was previously described.

### In Vitro Kinase Assay and Electrospray Ionization Mass Spectrometry

The Akt kinase assay was previously described. Purified full length BRCA1 protein was obtained from ProteinOne. pGEX-BRCA1 S694/T696 fusion protein was phosphorylated by Akt1 and prepared for mass spectrometry as previously described. Samples were concentrated to dryness and re-suspended in 1% formic acid to a total volume of 15 μL. The sample (2 μL) was injected onto a reverse-phase column using a cooled (8° C.) autosampler (Eksigent) connected to a HPLC system run at 14-18 μL/min before the split and ˜400 mL/min post-split (Eldex). A gradient of 5% to 30% acetonitrile over thirty two minutes was employed for peptide separation. Along with aqueous and organic washes, the total LC run time was sixty minutes. The column effluent was coupled directly via fused silica capillary transfer line to a QSTAR Pulsar™ Q-TOF tandem mass spectrometer (Sciex/Applied Biosystems) with a nanospray ion source. Data acquisition was performed using the instrument supplied Analyst software. The sixty minute LC runs were monitored by sequentially recording the precursor scan (MS, 1 s) followed by one collision-induced dissociation (CID) acquisitions (MS/MS, 4 s each). Singly charged ions were excluded from CID selection. Normalized collision energies were employed using nitrogen as the collision gas. The mass spectrometer control software Analyst™ was used to create de-isotoped centroided peak lists from raw spectra (.mgf format). These peak lists were searched against databases using an in-house developmental Protein Prospector™, LC Batch-Tag Web™ (Version 4.25.2, UCSF) and an in-house Mascot™ server (Version 2.0, Matrix Science). For searches mass tolerances were +/−100 ppm for MS peaks, and +/−0.3 Da for MS/MS fragment ions. Trypsin specificity was used allowing for 1 missed cleavage. The modifications of Met oxidation, protein N-terminal acetylation, peptide N-terminal pyro-glutamic acid formation and Ser, Thr, Tyr phosphorylation were allowed for. Samples were searched against all entries in the full NCBInr.

### Immunofluorescence Microscopy

Cells were transduced with the indicated adenovirus vectors at the following MOI's: Ad-BRCA1 and Ad-1853=25; Ad-myr-Akt and Ad-kd-Akt=100; Ad-LacZ at various MOI's to obtain a total MOI=125 for any individual transduction group. Cells were plated on cell culture grade glass cover slips (Fisher Scientific) and cultured in CSS medium for 48 hours prior to fixation with 10% neutral buffered formalin. Cells were permeabilized for 5 min. in 0.2% Triton X-100/PBS, washed in PBS, and then blocked in 2% BSA/PBS. Dual BRCA1 staining utilized an N-terminal mouse monoclonal antibody Ab-2 (1:50; Calbiochem) and an exon 11 directed rabbit polyclonal antibody (1:1250; BD Biosciences) diluted in blocking buffer. Anti-mouse 594 and anti-rabbit 488 (Molecular Probes) secondary antibodies were also diluted in blocking buffer. Nuclei were stained with DAPI (Sigma-Aldrich). Microscopic images were captured at 600× using a Nikon Eclipse 80i microscope and deconvolution was performed with Slidebook software (v4.1, Intelligent Imaging Innovations, Inc.).

### Colony Formation

Cells were transduced at a total MOI=110 in normal culture medium. MOI=10 for Ad-BRCA1 and Ad-1853. MOI=100 for Ad-myr-Akt and Ad-kd-Akt. Ad-GFP was used at various MOI's to achieve equal MOI's for all groups. Mock transduction groups were not exposed to adenovirus but were otherwise handled identically. 5000 cells per transduction were plated in triplicate in six-well plates. Cells were allowed to attach overnight and then each well was washed twice with PBS and incubated in CSS medium for 24 hours. Ionizing radiation treatment groups were exposed to 1, 2, or 4 Gy generated by a RS2000 irradiator (Rad Source Technologies, Inc.). All groups, including non-irradiated controls, were maintained in CSS medium for an additional 48 hours with medium refreshed at 24 hours. Then, all groups were changed back into normal culture medium and incubated for three weeks to allow colony outgrowth with medium changes every 3^{rd} day.

Cells were fixed in 10% neutral buffered formalin and stained with 0.2% crystal violet. Percentage survival for each group at each dose was determined by dividing the number of surviving colonies by the average number of colonies formed in the non-irradiated control for each transduction group (N=3). Error bars=SEM and a student's t-test was performed to determine significance (p<0.01).

### Sequence Alignment/Analysis

Human, mouse, rat, and chimpanzee sequences were accessed from Swis-Prot (#P38398, P48754, O54952, and Q9GKK8 respectively). Protein sequences were aligned with MegAlign software (v5.05, DNASTAR, Inc.) by the Clustal W method. Akt consensus recognition sequences were identified with Scansite (mit.scansite.edu).

### Example 1

### Estrogen Signaling Promoted Rapid BRCA1 Protein Accumulation

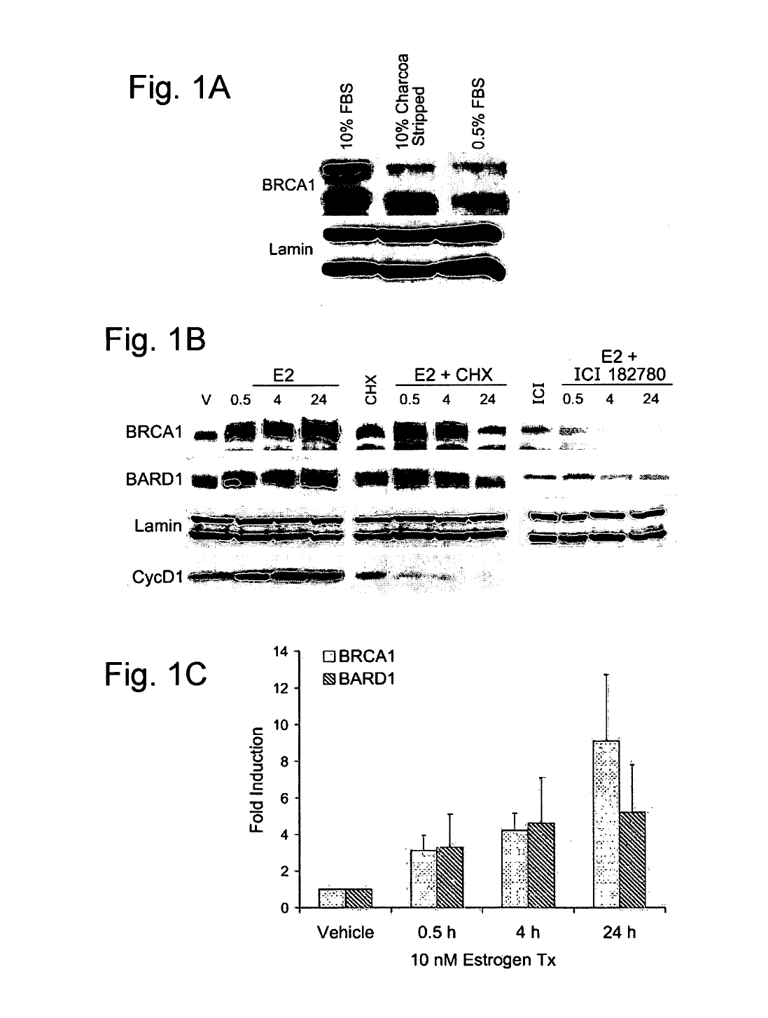

In one exemplary method, BRCA1 protein expression appeared significantly reduced in the ER positive MCF7 human breast carcinoma cell line when cultured in the absence of estrogen (FIG. 1(A), lane 2). The upper immunoreactive band in the BRCA1 panel represents full length BRCA1 (˜220 kD) while the lower band (˜180 kD) may represent a splice variant observed in some experiments. The loss of full length BRCA1 protein expression in steroid hormone depleted medium is equivalent to that observed in conditions of serum starvation suggesting that steroid signaling may play an important role in maintaining BRCA1 protein expression. Therefore, it was investigated whether estrogen (E2) stimulation of steroid depleted cultures could restore BRCA1 protein levels at early (0.5 hour), intermediate (4 hour), and extended (24 hour) times. E2 treatment consistently induced a two to four-fold increase of BRCA1 protein levels by one half hour (FIGS. 1B, 1C). A similar increase in BARD1 protein levels was also noted. Pretreatment with the ER antagonist ICI 182780 (Faslodex) abolished these increases, suggesting the effect is mediated through ER. However, pretreatment with the protein synthesis inhibitor cycloheximide (CHX) did not prevent the accumulation of BRCA1 and BARD1 at the early and intermediate time points (FIG. 1B), indicating that a post-translational mechanism that regulates protein stability could be involved.

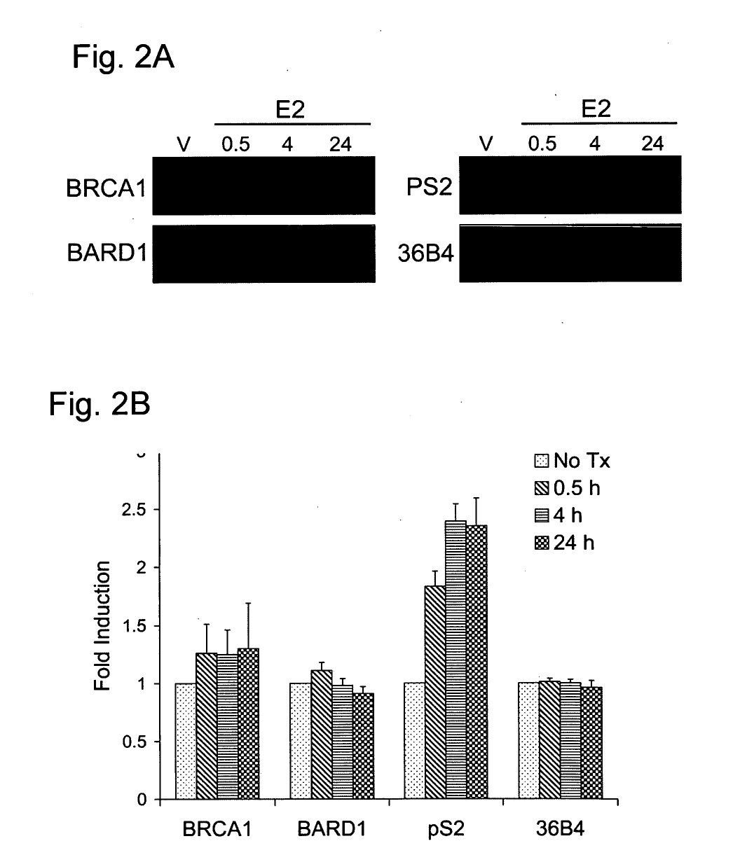

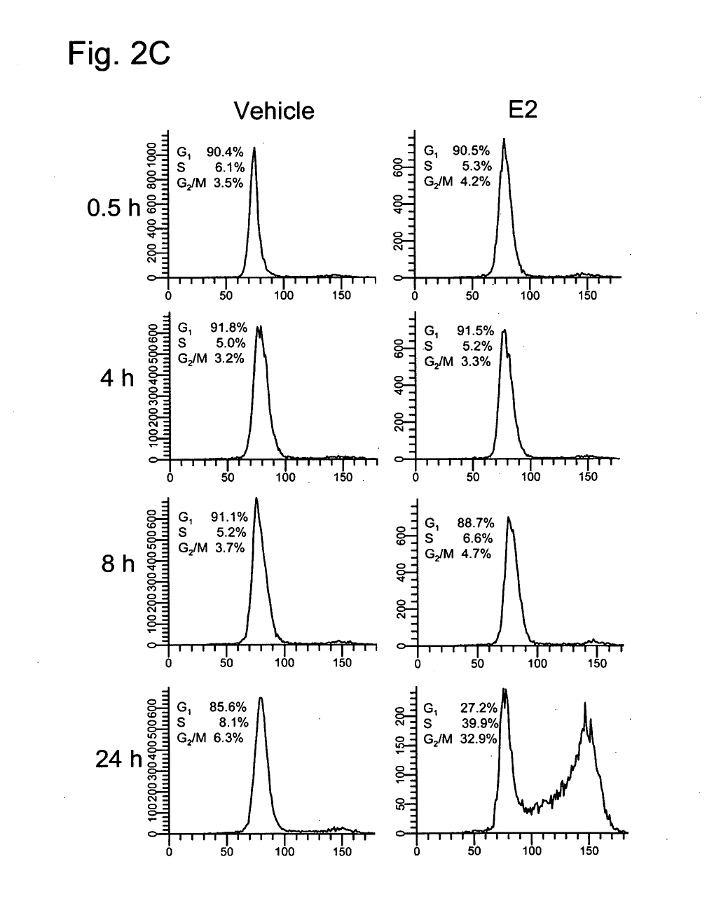

In another example, BRCA1 and BARD1 mRNA levels following E2 stimulation were also analyzed to confirm that the rapid accumulation of these proteins occurred independently of changes in transcription. RT-PCR demonstrated that BRCA1 and BARD1 mRNA levels did not change appreciably following E2 treatment in comparison to the estrogen responsive gene pS2, which served as a positive control (FIGS. 2A, 2B). BRCA1 protein levels are also modulated in a cell cycle dependent manner, with the highest levels observed in cells during S and G₂/M phases. The cell cycle distribution of MCF7 cells treated with either vehicle or E2 was analyzed to address whether the observed accumulation of BRCA1 was driven by cell cycle progression. In steroid depleted cell culture the majority of the population (>90%) is in G₁ (FIG. 2C), which may explain the very low levels of BRCA1 protein in these conditions. However, the rapid accumulation of BRCA1 and BARD1 proteins following E2 treatment observed at 0.5 and 4 hours (FIG. 1B) occurs well before there is significant progression of the cells into S phase (FIG. 2C). These results indicate that the rapid E2-dependent accumulation of BRCA1 and BARD1 proteins is not regulated by increased transcription or progression through the cell cycle.

### Example 2

### BRCA1 Protein Expression is Dependent on the PI3K/AKT Signaling Pathway

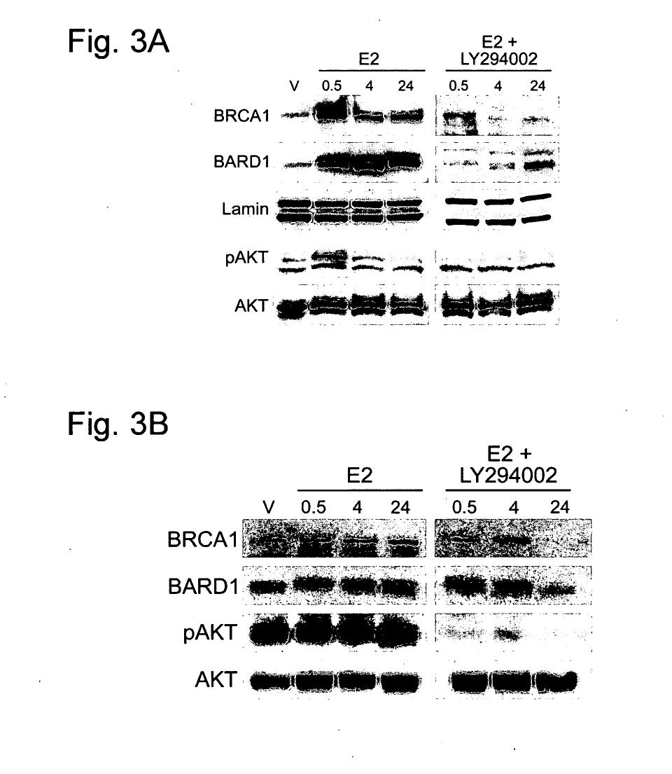

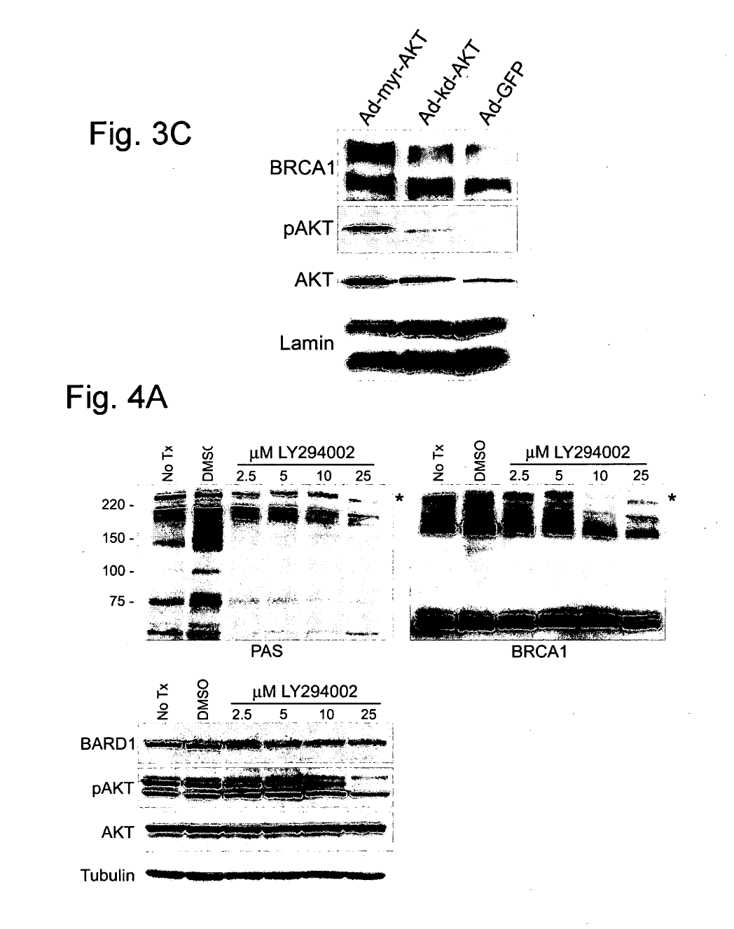

In another exemplary method, ability of several pathway specific inhibitors to prevent accumulation of BRCA1 were tested. Pretreatment with inhibitors of either MEK 1/2 or p38 MAP kinase had no effect on BRCA1 accumulation following E2 treatment. However, pretreatment with LY294002, which inhibits PI3K activity and prevents Akt activation, completely blocked the accumulation of both BRCA1 and BARD1 (FIG. 3A). Detection of phosphorylated Akt (pS473) indicated that Akt activation occurs rapidly after E2 stimulation concomitant with the increase of BRCA1 and BARD1 proteins. Blockade of Akt activation with LY294002, as evidenced by the lack of pS473-Akt signal, correlated with a lack of accumulation of BRCA1 and BARD1 proteins.

To ensure that this effect was not cell line specific, another ER+human breast carcinoma line, T47D, was tested. While the E2-stimulated accumulation of BRCA1 and BARD1 at early and intermediate time points did not appear as observed in MCF7 cells, treatment of T47D cells with E2+LY294002 resulted in decreased expression of BRCA1 and BARD1 proteins, recapitulating observations made with MCF7 cells (FIG. 3B). Analysis of Aktactivation indicated higher levels of both total and activated Akt in T47D cells cultured in steroid depleted medium compared to similarly cultured MCF7 cells, which may explain the difference in E2-regulated levels of BRCA1 and BARD1 protein in these two cell lines. Nevertheless, inhibition of PI3K and the subsequent decrease of phosphorylated Akt appeared correlated with decreased protein levels of both BRCA1 and BARD1 in T47D cells.

While inhibition of PI3K signaling implies that Akt could be involved, demonstration of definitive regulation of BRCA1 by Akt was desired. Recombinant human adenovirus expressing either a myristolated form of Akt (Ad-myr-Akt) which is constitutively activated, or a kinase dead mutant of Akt (Ad-kd-Akt) was utilized in this experiment. MCF7 cells were transduced with viruses encoding myr-Akt, kd-Akt, or GFP control protein, and only cells expressing myr-Akt were found to have elevated BRCA1 protein levels when cultured in the absence of E2 (FIG. 3C). This suggests that Akt may directly regulate BRCA1 protein stability.

### Example 3

### Akt Regulates BRCA1 Protein Levels Through Direct Phosphorylation

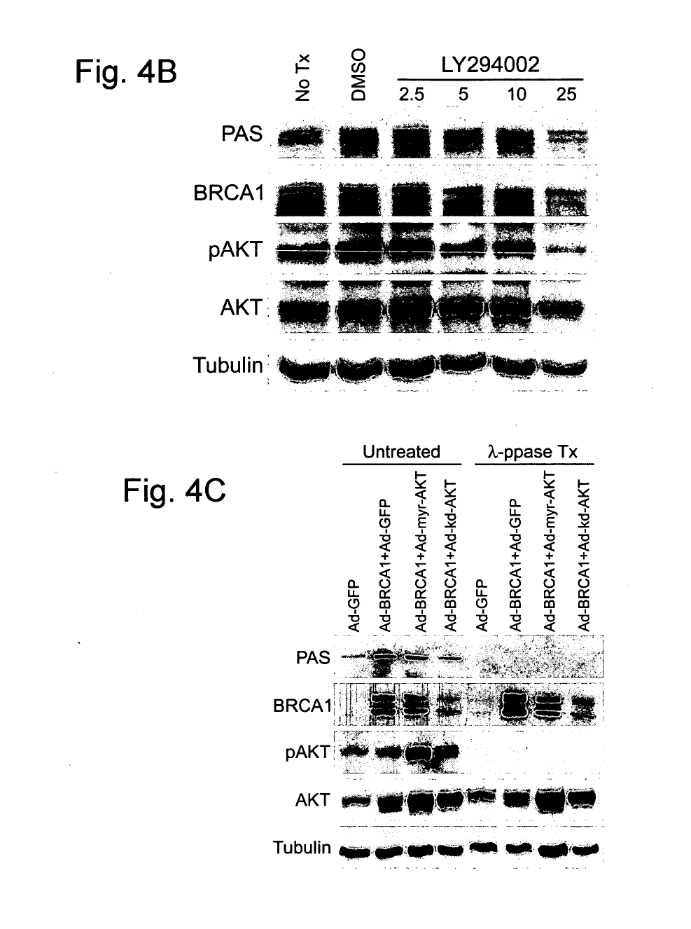

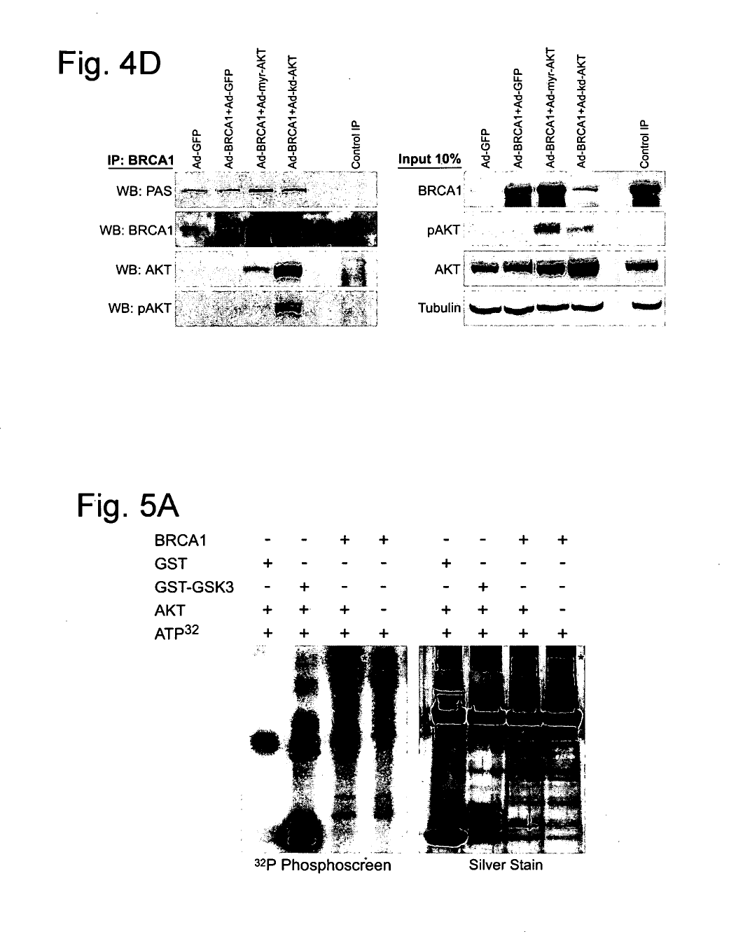

In another exemplary method, because constitutively activated Akt was sufficient to restore levels of BRCA1 protein expression in steroid depleted culture of MCF7 cells, whether Akt was directly phosphorylating BRCA1 was investigated. Whole cell lysates were prepared from MCF7 cells cultured in medium containing normal fetal bovine serum and varying doses of LY294002. Cell lysates were immunoblotted using a polyclonal antibody to the phosphorylated form of the Akt substrate consensus sequence R-X-R-X-X-(pT/pS) (PAS antibody, SEQ ID NO:9) demonstrating a strong immunoreactive phospho-Akt substrate which decreased in a dose dependent manner with LY294002 treatment (FIG. 4A). A monoclonal antibody to BRCA1 reacted with a substrate of the same molecular weight that similarly decreased in a LY294002 dose dependent manner. Total BARD1 protein levels were found to decrease in a dose responsive manner as well, and the immunoblot for pAkt confirmed that Akt activation was indeed inhibited (FIG. 4A). This experiment was repeated in T47D cells and obtained similar results (FIG. 4B).

In another exemplary experiment to investigate the interaction between BRCA1 and Akt, exogenous BRCA1 with myr-Akt, kd-Akt, or a GFP control protein using adenoviral vectors was expressed. This experiment demonstrated that the kd-Akt protein noticeably antagonized expression of exogenous BRCA1 in comparison with either myr-Akt or control (FIG. 4C), lanes 2-4. Small amounts of BRCA1 expressed in these cells reacted with the PAS antibody, indicating that this protein may have been phosphorylated by endogenous, wild-type Akt. In comparison, exogenous BRCA1 protein levels were approximately equal in cells transduced with Ad-BRCA1 and either Ad-myr-Akt or Ad-GFP control, suggesting that over-expression of BRCA1 from an adenoviral vector can overcome the normal degradation mechanism with only minimal activation of endogenous Akt being required. Additionally, it was found that BRCA1 from cell lysates treated with λ-phosphatase were not recognized by the PAS antibody compared with untreated samples, demonstrating the antibody's specificity for a phosphorylated epitope (FIG. 4C), lanes 6-8. Immunoprecipitation from similarly transduced cells with BRCA1 antibodies followed by immunoblot with the PAS antibody demonstrated an immunoreactive band at ˜220 kD, confirming the identity of this substrate as BRCA1 (FIG. 4D). Further, Akt co-immunoprecipitated with BRCA1 demonstrating an interaction between these two proteins, which is frequently observed with other substrates for Akt. Significantly higher amounts of kd-Akt were co-immunoprecipitated with BRCA1 in kd-Akt expressing cells compared to those expressing myr-Akt, suggesting that the kd-Akt may bind to BRCA1 but may not efficiently release it due to the lack of kinase activity. In total, the results from immunoblot studies with the PAS antibody strongly suggest that Akt phosphorylates BRCA1 in vivo and this phosphorylation appears to contribute to Akt regulation of BRCA1 protein levels.

### Example 4

### Akt Directly Phosphorylates BRCA1 In Vitro at Both 5694 and T509

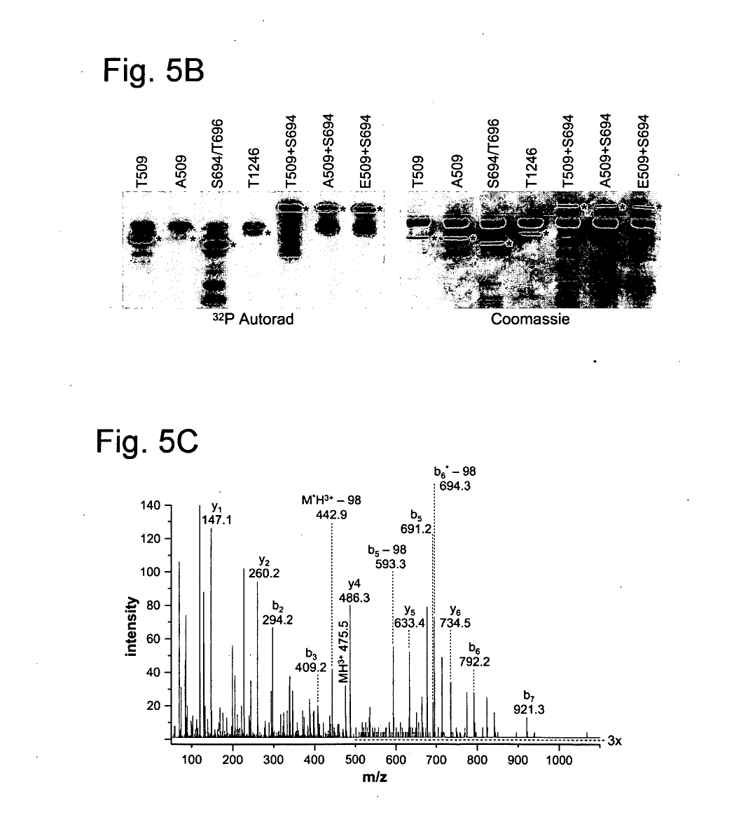

To further examine apparent direct phosphorylation of BRCA1 by Akt, in vitro kinase reactions were performed using purified full length BRCA1 and purified active Akt1. FIG. 5A demonstrates that full length BRCA1 is phosphorylated by Akt in vitro. This finding is consistent with a previous report that suggested full length BRCA1 was phosphorylated by Akt and identified T509 as the target residue in GST fusion proteins. To investigate the possibility of additional sites of phosphorylation, human and mouse BRCA1 protein sequences were analyzed using Scansite (http://scansite.mit.edu) for other possible Akt consensus sites which appeared to be conserved. Three potential Akt consensus sites of moderate probability were identified using this program: the previously identified T509, T696, and T1246. However, it was noted that T696 was not conserved in the aligned mouse sequence, but nearby 5694 of human BRCA1 was conserved and was also identified by Scansite as a potential Akt substrate. GST-fusion proteins were constructed containing approximately 200 amino acids spanning T509, S694/T696, or T1246 and these purified fusion proteins were utilized in an in vitro Akt kinase assay. Results of this experiment showed that both the T509 and S694/T696 constructs were strongly phosphorylated, while T1246 did not appear to be phosphorylated (FIG. 5B), lanes 1-4. A fusion protein with a T509A mutation was not phosphorylated, indicating that T509 is the site of phosphorylation in this construct. The S694/T696 fusion protein was then analyzed by electrospray mass spectrometry and identified the conserved S694 residue as the site of phosphorylation by Akt (FIG. 5C).

Next, 400 amino acid segments of human BRCA1 fused to GST were created which encompassed both the T509 and S694 residues. In addition, T509A mutant and phospho-mimic T509E mutant constructs were created which also contained the S694 site. Both A509+S694 and E509+S694 constructs were equally phosphorylated by Akt, with the intensity of these signals approximately equal to one half of the signal obtained with the wild-type T509+S694 construct. This result indicates that the identity of amino acid 509 does not affect the efficiency of phosphorylation at 5694 in vitro (FIG. 5B).

### Example 5

### Proteasome Inhibition Results in Rapid Accumulation of BRCA1 Protein

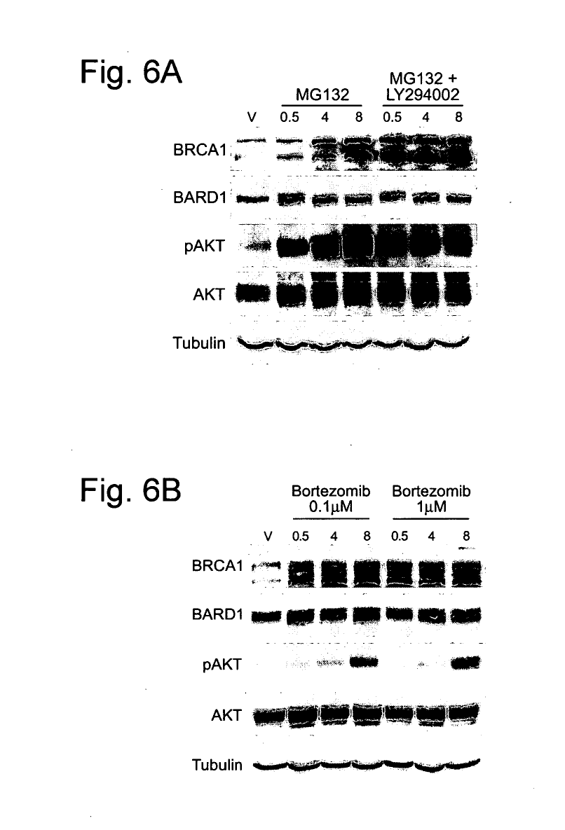

In another example, rapid accumulation of BRCA1 and BARD1 proteins following E2 stimulation and Akt activation appeared to occur independently of translation. Therefore it was sought to determine if constitutive degradation of BRCA1 and BARD1 by the 26S proteasome was responsible for keeping these protein levels very low in cells cultured in the absence of steroid hormones. MCF7 cells cultured in steroid depleted medium were treated with the proteasome inhibitor MG132 for either 0.5, 4, or 8 hours. This treatment resulted in accumulation of both BRCA1 and BARD1 proteins beginning within a half hour after the addition of MG132 (FIG. 6A). It was noted in preliminary experiments that MG132 treatment led to dramatic activation of Akt, confounding whether the accumulation of BRCA1 and BARD1 was due to proteasome inhibition or via indirect activation of Akt. Therefore, cells were co-treated with LY294002 and MG132 to prevent Akt activation, and found that BRCA1 and BARD1 protein levels increased rapidly under these conditions as well. This result confirmed that prevention of proteasome-mediated degradation of BRCA1 and BARD1 appears to be downstream of Akt activity. It was also tested whether a clinically utilized proteasome inhibitor, bortezomib (e.g. Velcade), could restore BRCA1 and BARD1 protein in a similar experiment. Both doses of bortezomib rapidly increased BRCA1 and BARD1 expression, and only minor activation of Akt was observed by 8 hours (FIG. 6B). These results demonstrated that treatment with two individual proteasome inhibitors rapidly increases BRCA1 and BARD1 protein levels similar to results observed after estrogen treatment.

### Example 6

### Akt Activity Appears to Support Nuclear Accumulation of BRCA1

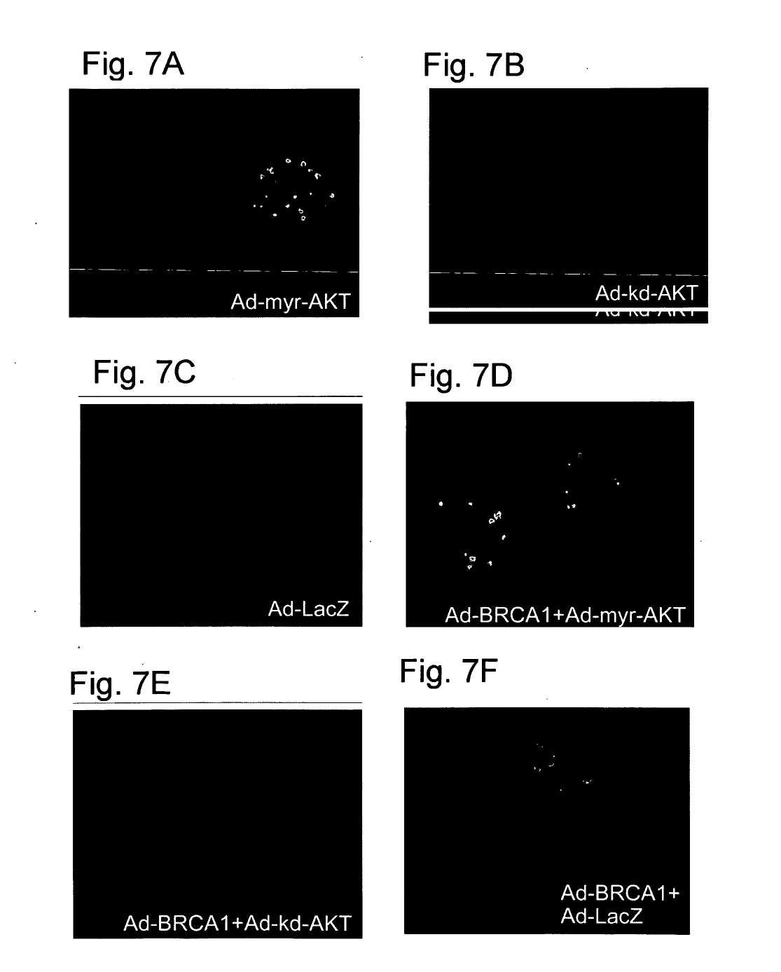

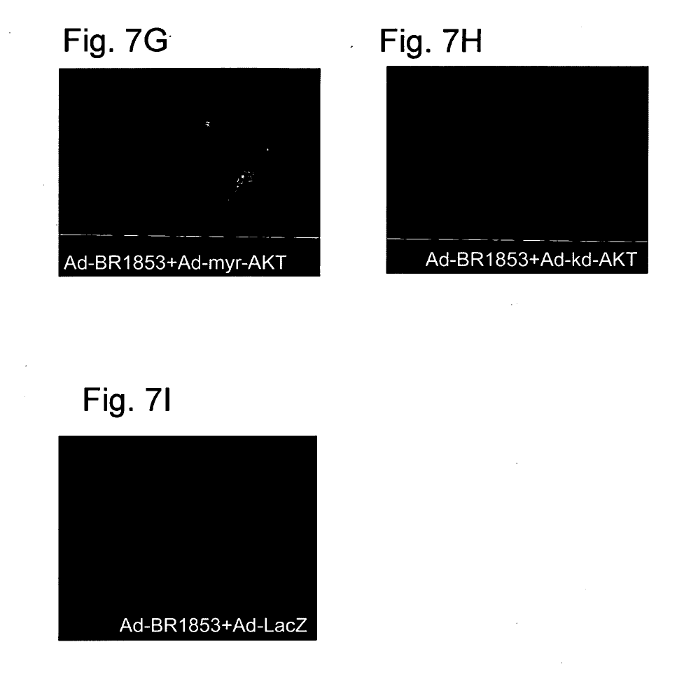

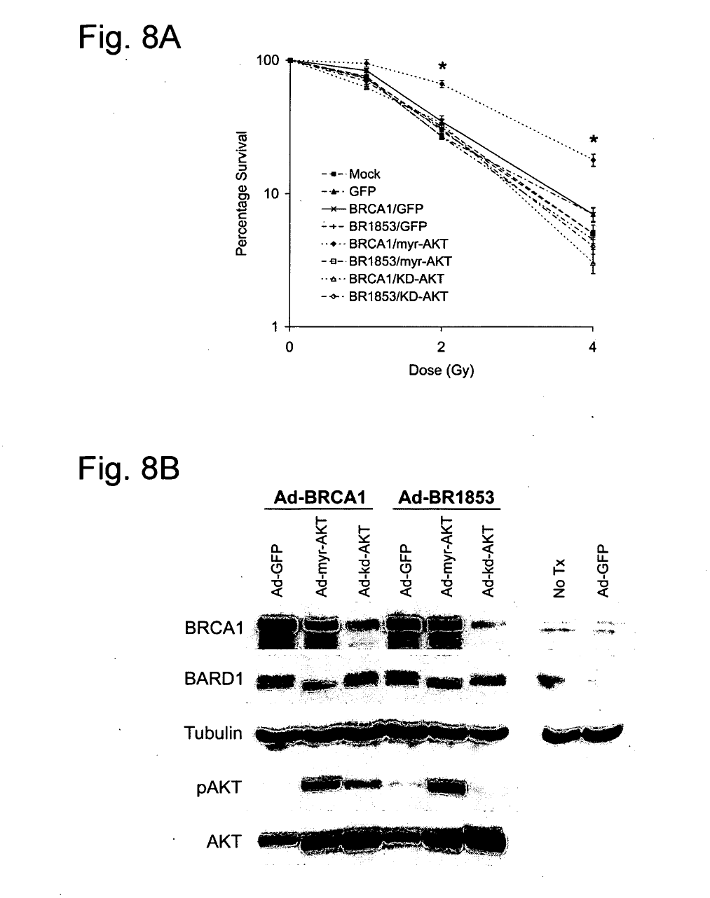

BRCA1 is a nuclear localized protein that appears to form discrete foci during S-phase and following DNA damage. Mutations within the BRCT domain, including a common truncation at amino acid 1853, result in mislocalization of BRCA1 to the cytoplasm. Furthermore, it was recently demonstrated that a BRCA1 T509A protein was inefficiently transported into the nucleus, suggesting that Akt might regulate its nuclear translocation. To address whether Akt activity affected BRCA1 subcellular localization, immunofluorescence studies were performed on MCF7 cells cultured in steroid depleted medium which had been transduced with adenoviral vectors expressing either wild-type (Ad-BRCA1) or a truncated (Ad-BR1853) BRCA1 protein in combination with activated Akt (Ad-myr-Akt), kinase dead Akt (Ad-kd-Akt), or control vector (Ad-LacZ). Cells were stained with two independent anti-BRCA1 antibodies to confirm specificity of the staining for BRCA1. Kinase dead Akt had a dominant negative effect on BRCA1 levels and these cells showed decreased overall staining (FIGS. 7E and 7H), consistent with immunoblot data (FIGS. 4C and 4D). In addition, when cells with specific staining were observed, BRCA1 appeared to be localized in the cytoplasm (FIG. 8E) suggesting that kd-Akt antagonized nuclear localization of the protein. Expression of myr-Akt alone stimulated focal nuclear staining for BRCA1 in a proportion of cells, compared to an almost total lack of specific staining in cells transduced with kd-Akt or control (FIGS. 7A-7C). Co-expression of wild-type BRCA1 with myr-Akt stimulated focal nuclear staining, and the intensity of staining observed in cells expressing both BRCA1 and myr-Akt was far greater than that observed in control cells expressing BRCA1 plus β-gal (FIGS. 7D and 7F). Expression of the BRCT-truncated mutant BRCA1 (1853stop) yielded primarily cytoplasmic staining in all instances (FIGS. 7G, 7H and 7I), consistent with the previous report that the BRCT domain is necessary for nuclear localization. Taken together, these data imply that Akt function plays a role in facilitating nuclear localization of BRCA1. However, activated Akt is not sufficient to drive the nuclear accumulation of a truncated BRCA1 protein, indicating that additional sequences within BRCA1 are required for nuclear translocation.

### Example 7

### Co-Expression of Activated Akt and Wild-Type BRCA1 Improves Radiation Survival

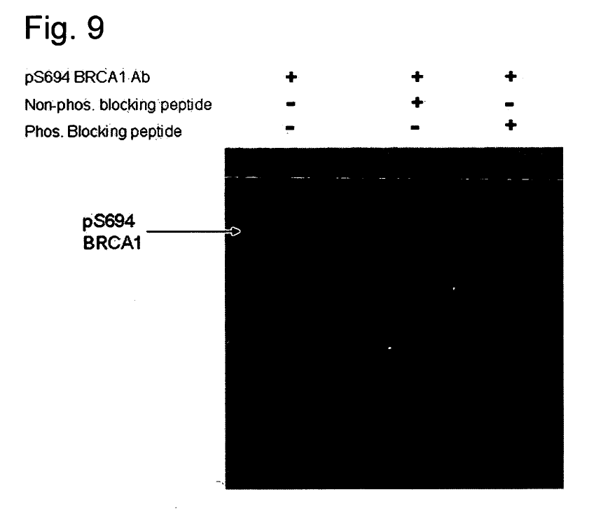

Loss of BRCA1 function results in hypersensitivity to ionizing radiation. It was hypothesized that the low levels of BRCA1 and BARD1 protein expressed in MCF7 cells cultured under steroid depleted conditions would result in increased sensitivity to radiation treatment. Further, it was predicted that the stabilization of exogenous BRCA1 by activated Akt would decrease radiation sensitivity. To test this hypothesis, cells were transduced with either wild-type or truncated (1853stop) BRCA1 protein in combination with myr-Akt, kd-Akt, or GFP control. These groups were cultured in steroid depleted medium, exposed to 0, 1, 2, or 4 Gy of ionizing radiation under these conditions, and later returned to normal culture medium for colony outgrowth which was quantitated at three weeks. Cells co-expressing wild-type BRCA1 and myr-Akt appeared to demonstrate significantly improved survival compared to other groups (FIG. 8A). The group expressing truncated BRCA1 (1853stop) plus myr-Akt showed no improvement in survival compared to control groups, indicating that the pro-survival activity of myr-Akt without wild-type BRCA1 is not sufficient to increase colony formation. Further, cells which expressed wild-type BRCA1 with control vector did not show a statistically significant improvement in survival. Immunoblots of parallel cultures harvested at the time of irradiation demonstrated that wild-type BRCA1 was expressed at approximately equal levels in Ad-BRCA1 groups co-expressing either myr-Akt or GFP control (FIG. 9(B), similar to results discussed above (FIG. 4(C). BARD1 levels were also comparable in these two groups. Therefore, the improved survival of cells expressing BRCA1 with myr-Akt compared to those expressing BRCA1 with control GFP suggests that Akt may positively support the role of BRCA1 in the regulation of DNA repair in addition to stabilizing its expression.

### Example 8

### Generation and Application of a Phosphospecific Antibody to Detect BRCA1 Protein which is Specifically Phosphorylated on Serine 694

A rabbit polyclonal antibody that binds a BRCA1 protein molecule phosphorylated on serine 694 has been developed and demonstrated that it detects a 220 kD protein with properties identical to BRCA1 (FIG. 9). The antisera was generated by immunizing rabbits using the phosphorylated peptide TSKRHDphosphoS-DTFPELK (SEQ ID NO:10) to immunize and then phosphospecific antisera was obtained by purifying the resulting antisera against nonphospho TSKRHDSDTFPELK-sepharose (SEQ ID NO:11) (to remove antibodies not specific for the phosphorylated protein) and then eluting the antisera that binds specifically to the phosphorylated peptide. The antisera was tested as shown (FIG. 9). This figure illustrates that a specific antibody can be produced to a phosphorylated molecule and used as proposed in some embodiments of the present invention.

All of the COMPOSITIONS and METHODS disclosed and claimed herein can be made and executed without undue experimentation in light of the present disclosure. While the compositions and methods have been described in terms of preferred embodiments, it is apparent to those of skill in the art that variations may be applied to the COMPOSITIONS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. A composition comprising:
an antibody or an antibody-fragment directed to bind phosphorylated threonine 509 or phosphosphorylated serine 694 of a polypeptide sequence of BRCA1 (SEQ ID NO:12)

2. The composition of claim 1, wherein the antibody or antibody-fragment comprises an anti-phosphosphorylated serine 694 antibody of a polypeptide sequence of all or part of BRCA1 derived sequence SEQ ID NO:10.

3. The composition of claim 1, wherein the anti-phosphorylated serine 694 antibody is a polyclonal antibody.

4. The composition of claim 1, wherein the anti-phosphosphorylated serine 694 antibody is a monoclonal antibody.

5. A method of treating cancer in a subject comprising;
identifying a subject having cancer;
identifying whether the subject has an unstable form of BRCA1; and
administering to the subject having unstable BRCA1, a composition comprising a therapeutically effective amount of an Akt activator, or a pharmaceutically acceptable salt thereof wherein.

6. The method of claim 5, wherein the Akt activator is selected from the group consisting of estrogen, IGF-I, calcium/calmodulin, insulin, PtdIns-3.4,-P2, Ro 31-8220 or combination thereof.

7. The method of claim 5, wherein the subject has a low level of BRCA1 protein compared to a control level of BRCA1 protein.

8. The method of claim 5, wherein the cancer is selected from the group consisting of breast cancer, ovarian cancer and prostate cancer.

9. The method of claim 5, further comprising administering to the subject a composition comprising a therapeutically effective amount of at least one of a protease inhibitor and a proteosome inhibitor.

10. The method of claim 5, wherein the cancer is an inherited cancer.

11. The method of claim 5, wherein the Aid activator increases stabilization of BRCA1.

12. The method of claim 5, wherein the Akt activator increases phosphorylation of BRCA1.

13. The method of claim 5, wherein the identification of unstable BRCA1 protein comprises:
collecting a biological sample from the subject;
contacting the sample with one or more of an anti-phosphosphorylated threonine 509 antibody or an anti-phosphorylated serine 694 antibody of BRCA1; and
determining level of binding of the one or more antibodies to the sample, wherein a low level of binding of the one or more antibodies in the sample compared to a control level of binding a control sample is an indication that the subject has unstable BRCA1 protein.

14. A kit comprising;
a composition comprising an antibody or antibody fragment directed to phosphorylated threonine 509 or phosphorylated serine 694 of BRCA1; and
one or more suitable containers.

15. The kit of claim 14, wherein the antibody or antibody fragment comprises an anti-phosphorylated serine 694 antibody or antibody fragment of BRCA1 derived sequence TSKRHDphosphoS-DTFPELK (SEQ ID NO:10).

16. The kit of claim 14, wherein the antibody or antibody fragment is selected from the group consisting of:
(a) a monoclonal antibody;
(b) a humanized antibody;
(c) a human monoclonal antibody;
(d) a subhuman primate antibody; and
(e) an antibody fragment derived from (a), (b), (c) or (d).
